(19) United States
(12) Patent Application Publication
(10) Pub. No.: US 2024/0009154 A1
(43) Pub. Date: Jan. 11, 2024

(54) METHODS AND COMPOSITIONS FOR TREATING CYTOKINE RELEASE SYNDROME

(71) Applicant: Temple University-Of The Commonwealth System of Higher Education, Philadelpnia, PA (US)

(72) Inventors: Mark Feitelson, North Wales, PA (US); Alla ARZUMANYAN, Huntington Valley, PA (US); lra Spector, Jenkintown, PA (US)

(21) Appl. No.: 18/040,515

(22) PCT Filed: Aug. 4, 2021
(86) PCT No.: PCT/US2021/044455

§ 371 Date: Feb. 3, 2023

Related U.S. Application Data
(60) Provisional application No. 63/060,912, filed on Aug. 4, 2020.
(60) Provisional application No. 63/060,920, filed on Aug. 4, 2020.

(51) Int. Cl. A61K 31/19; A61K 47/38; A61K 45/06; A61P 37/06

(52) U.S. Cl. CPC A61K 31/19; A61K 47/38; A61K 45/06; A61P 37/06

(57) ABSTRACT

Disclosed herein are methods and pharmaceutical compositions to treat systemic inflammatory response syndrome. In some embodiments, a pharmaceutical composition disclosed herein comprises at least two short chain fatty acids or pharmaceutically-acceptable salts thereof in a ratio of at least 4:1. In some embodiments, administering a pharmaceutical composition of the disclosure reduces a level of a cytokine in the subject.

### CROSS-REFERENCE

This application claims the benefit of U.S. Provisional Application No. 63/060,912, filed Aug. 4, 2020; and U.S. Provisional Application No. 63/060,920, filed Aug. 4, 2020, each of which are incorporated herein by reference in their entirety.

### BACKGROUND

Acute respiratory distress syndrome is characterized by an over-reactive immune response, which is associated with a sustained production of high levels of pro-inflammatory cytokines. The sustained production of cytokines, also known as cytokine release syndrome, can cause extensive damage to lungs and increase the risk for secondary infections within the lung. Bacterial metabolites that exhibit strong inflammatory properties can be used to treat acute respiratory distress syndrome.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### SUMMARY OF THE INVENTION

In some embodiments, described herein is a method of treating a condition comprising: administering to a subject in need thereof a pharmaceutical composition comprising a therapeutically-effective amount of a first short chain fatty acid or a pharmaceutically-acceptable salt thereof and a therapeutically-effective amount of a second short chain fatty acid or a pharmaceutically-acceptable salt thereof, wherein the first short chain fatty acid or the pharmaceutically-acceptable salt thereof and the second short chain fatty acid or the pharmaceutically-acceptable salt thereof are present in the formulation in a ratio of at least 10:1.

In some embodiments, described herein is a method of treating a condition comprising: administering to a subject in need thereof a pharmaceutical composition comprising a therapeutically-effective amount of a first short chain fatty acid or a pharmaceutically-acceptable salt thereof and a therapeutically-effective amount of a second short chain fatty acid or a pharmaceutically-acceptable salt thereof, wherein the administering reduces a level of a cytokine in the subject.

In some embodiments, described herein is a method of treating a condition comprising: administering to a subject in need thereof a pharmaceutical composition comprising a therapeutically-effective amount of a first short chain fatty acid or a pharmaceutically-acceptable salt thereof and a therapeutically-effective amount of a second short chain fatty acid or a pharmaceutically-acceptable salt thereof, wherein the administering modulates an effect of the subject's immune system.

In some embodiments, described herein is a pharmaceutical composition comprising: a) a first short chain fatty acid or a pharmaceutically-acceptable salt thereof; and b) a second short chain fatty acid or a pharmaceutically-acceptable salt thereof, wherein the first short chain fatty acid or the pharmaceutically-acceptable salt thereof and the second short chain fatty acid or the pharmaceutically-acceptable salt thereof are present in the formulation in a ratio of at least 10:1.

In some embodiments, described herein is a pharmaceutical composition comprising: a) butyrate or a pharmaceutically-acceptable salt thereof; and b) propionate or a pharmaceutically-acceptable salt thereof; wherein butyrate or the pharmaceutically-acceptable salt thereof and propionate or the pharmaceutically-acceptable salt thereof are present in the pharmaceutical composition in a ratio of at least 10:1.

### BRIEF DESCRIPTION OF THE DRAWINGS

FIG. 1 shows plasma IL-6 levels measured by ELISA.

FIG. 2 shows plasma TNF-α levels measured by ELISA.

### DETAILED DESCRIPTION OF THE INVENTION

Cytokines are a group of small proteins that are secreted by cells for intracellular signaling and communication. Specific cytokines can exhibit autocrine, paracrine, or endocrine activity. By binding to receptors, cytokines can elicit a variety of responses, depending upon the cytokine and the target cell. Among the many functions of cytokines are the control of cell proliferation and differentiation, regulation of angiogenesis, and regulation of immune and inflammatory responses. VEGF (angiogenesis) promotes inflammation by increasing capillary permeability and influx of both cells and cytokines into damaged tissue. Short chain fatty acids (SCFAs) can block angiogenesis.

The major types of cytokines include interferons, interleukins, colony-stimulating factors, and tumor necrosis factors. Interferons act to regulate innate immunity and to activate antiviral properties and antiproliferative effects. Interleukins promote growth and differentiation of leukocytes, and chemokines control chemotaxis and leukocyte recruitment. Colony-stimulating factors stimulate hematopoietic progenitor cell proliferation and differentiation, and tumor necrosis factors are proinflammatory and activate cytotoxic T lymphocytes.

Cytokine release syndrome (CRS), cytokine storm syndrome (CSS), or a cytokine storm, is a form of systemic inflammatory response syndrome (SIRS). CRS occurs when the immune system is fighting pathogens, such as an infectious or noninfectious disease. CRS can also occur as an adverse effect of administering some monoclonal antibody drugs and adoptive T-cell therapies.

Immune cells are activated by stressed or infected cells through receptor-ligand interactions. Cytokines produced by immune cells recruit more effector immune cells such as T-cells and inflammatory monocytes to the site of inflammation or infection. Pro-inflammatory cytokines bind cognate receptors on immune cells, resulting in the activation and stimulation of further cytokine production. A dysregulated inflammatory response can be life-threatening due to systemic hyperinflammation, hypotensive shock, and multi-organ failure.

Inflammation associated with CRS begins at a local site and spreads throughout the body via the systemic circulation. Symptoms of CRS include fever, fatigue, loss of appetite, muscle and joint pain, nausea, vomiting, diarrhea, rashes, fast breathing, rapid heartbeat, low blood pressure, seizures, headache, confusion, delirium, hallucinations, tremor, and loss of coordination. Lab tests and clinical monitoring show low blood oxygen, widened pulse pressure, increased cardiac output for early CRS, diminished cardiac output for late CRS, high levels of nitrogen compounds in the blood, elevated D-dimer levels, elevated transaminases, factor I deficiency and excessive bleeding, or increased levels of bilirubin.

Acute respiratory distress syndrome (ARDS), which can result from CRS, is a lung condition that causes low blood oxygen and is characterized by rapid onset of widespread inflammation in the lungs. Patients who develop ARDS are usually ill due to another disease or a major injury. The underlying mechanism involves diffuse injury to cells that form the barrier of the microscopic air sacs of the lungs, surfactant dysfunction, activation of the immune system, and dysfunction of the body's regulation of blood clotting. Fluid builds up inside the air sacs of the lungs, and surfactant breaks down. Surfactant is a foamy substance that keeps the lungs fully expanded so that a person can breathe. Changes resulting from ARDS prevent the lungs from filling properly with air and moving enough oxygen into the bloodstream and throughout the body. The lung tissue can scar and become stiff. ARDS can develop over a few days, or can get worse very quickly. Patients are also at increased risk for developing secondary respiratory infections, which contribute to morbidity and mortality among ARDS patients.

Symptoms of ARDS include shortness of breath, rapid breathing, a fast heart rate, coughing that produces phlegm, blue fingernails or blue tone to the skin or lips, fatigue, fever, crackling sound in the lungs, chest pain, low blood pressure, and confusion. ARDS can be accompanied by atelectasis (i.e., collapse of small air pockets in the lungs), blood clots forming from lying down for long periods, weakness in muscles used for breathing or moving around, infections, stress ulcers, depression, or other mood disorders. ARDS can also be accompanied by multiple organ failure or pulmonary hypertension.

SCFAs are saturated aliphatic acids consisting of one polar carboxylic acid moiety and a hydrophobic hydrocarbon chain. SFCAs, which are bacterial metabolites, have anti-inflammatory properties and reduce cytokine storms, providing extra time for infected people to develop immunity to a virus in the absence of disease.

EGFR signaling can be proinflammatory via activation of MAPK and NF-κB. PI3K is involved in NF-κB activation and is proinflammatory. Ras signaling stimulates expression of the pro-inflammatory IL-8. Wnt signaling can be pro- or anti-inflammatory depending upon the signal's ability to cross-talk with NF-κB. Suppression of NF-κB by SCFAs also results in the down-regulated expression of cytokines, which, in turn, down-regulates angiogenesis. FGF and IGF signaling can be pro- or anti-inflammatory. Heterotrimeric G-protein signaling is also strongly proinflammatory. PDGF signaling is associated with chronic inflammation while decreased PDGF is associated with reduced inflammation. Toll-like receptor signaling results in innate, proinflammatory responses. Thus, down-regulated expression of the signaling pathways by SCFAs is useful in ameliorating, delaying, preventing, or lessening a likelihood or severity of cytokine storm syndrome.

Disclosed herein are pharmaceutical compositions comprising at least one SCFA to treat, delay, or lessen the severity of, or block the development of CRS. Disclosed herein are pharmaceutical compositions comprising at least one SCFA to treat, delay, lessen the severity of, block the development of, or reduce a likelihood of developing ARDS. The disclosed pharmaceutical compositions can give infected patients more time to develop neutralizing antibodies against a virus with developing disease. Also disclosed herein are methods of using pharmaceutical compositions comprising at least one SCFA to treat, delay, lessen the severity of, block the development of, or reduce a likelihood of developing CRS. Further, disclosed herein are methods of using pharmaceutical compositions comprising at least one SCFA to treat, delay, lessen the severity of, block the development of, or reduce a likelihood of developing ARDS.

In some embodiments, described herein is a method of treating a condition comprising: administering to a subject in need thereof a pharmaceutical composition comprising a therapeutically-effective amount of a first short chain fatty acid or a pharmaceutically-acceptable salt thereof and a therapeutically-effective amount of a second short chain fatty acid or a pharmaceutically-acceptable salt thereof, wherein the first short chain fatty acid or the pharmaceutically-acceptable salt thereof and the second short chain fatty acid or the pharmaceutically-acceptable salt thereof are present in the formulation in a ratio of at least 10:1. In some embodiments, the administering reduces a level of a cytokine in the subject.

In some embodiments, described herein is a method comprising: administering to a subject in need thereof a pharmaceutical composition comprising a therapeutically-effective amount of a first short chain fatty acid or a pharmaceutically-acceptable salt thereof and a therapeutically-effective amount of a second short chain fatty acid or a pharmaceutically-acceptable salt thereof, wherein the first short chain fatty acid or the pharmaceutically-acceptable salt thereof and the second short chain fatty acid or the pharmaceutically-acceptable salt thereof are present in the formulation in a ratio of at least 10:1. In some embodiments, the administering modulates an effect of the subject's immune system. In some embodiments, the administering reduces a level of a cytokine in the subject.

In some embodiments, described herein is a pharmaceutical composition comprising: a) a first short chain fatty acid or a pharmaceutically-acceptable salt thereof; and b) a second short chain fatty acid or a pharmaceutically-acceptable salt thereof, wherein the first short chain fatty acid or the pharmaceutically-acceptable salt thereof and the second short chain fatty acid or the pharmaceutically-acceptable salt thereof are present in the formulation in a ratio of at least 10:1.

In some embodiments, described herein is a pharmaceutical composition comprising: a) butyrate or a pharmaceutically-acceptable salt thereof; and b) propionate or a pharmaceutically-acceptable salt thereof; wherein butyrate or the pharmaceutically-acceptable salt thereof and propionate or the pharmaceutically-acceptable salt thereof are present in the pharmaceutical composition in a ratio of at least 10:1.

### Mechanism of Action

SCFAs are the main metabolic products of anaerobic bacteria fermentation in the intestine. SCFAs affect cellular processes, such as gene expression, chemotaxis, differentiation, proliferation, and apoptosis via activation of G protein coupled receptors (GPCRs), inhibition of histone deacetylases (HDACs), and binding to butyrate-responsive elements in the gene promoter regions of transcriptional factors. SCFAs regulate inflammation by controlling migration of immune cells toward inflammatory sites and by modulating the activation state of immune cells. SCFAs also influence the balance between pro-inflammatory and anti-inflammatory cells and facilitate communication between microbiota and the immune system. The principle mechanisms through which SCFAs exert anti-inflammatory effects are suppression of TNFα and NF-κB activation, inhibition of IFN-γ production, and upregulation of the peroxisome proliferator-activated receptor γ (PPARγ).

### Compounds of the Disclosure

SCFAs are saturated aliphatic acids consisting of one polar carboxylic acid moiety and a hydrophobic hydrocarbon chain. The present disclosure describes use of an SCFA, an SCFA precursor, an SCFA biosynthesis precursor, a compound comprising an SCFA moiety, an SCFA derivative, or a pharmaceutically-acceptable salt thereof.

In some embodiments, a compound of the disclosure is formic acid, acetic acid, propionic acid, butyric acid, isobutyric acid, valeric acid, or isovaleric acid. In some embodiments, a compound of the disclosure is formate, acetate, propionate, butyrate, isobutyrate, valerate, or isovalerate, or a pharmaceutically-acceptable salt thereof. In some embodiments, a compound of the disclosure is sodium formate, sodium acetate, sodium propionate, sodium butyrate, sodium isobutyrate, sodium valerate, or sodium isovalerate. In some embodiments, a compound of the disclosure is methoxyacetic acid, valproic acid, 3-methoxypropionic acid, ethoxyacetic acid, tributyrin, or propionate ester. In some embodiments, a compound of the disclosure is butyrate, N-acetylbutyrate, phenylbutyrate, isobutyrate, pivaloyloxymethylbutyrate, or monoacetone glucose-3-butyrate. In some embodiments, a compound of the disclosure is sodium butyrate, sodium N-acetylbutyrate, sodium phenylbutyrate, sodium isobutyrate, sodium pivaloyloxymethylbutyrate, or sodium monoacetone glucose-3-butyrate.

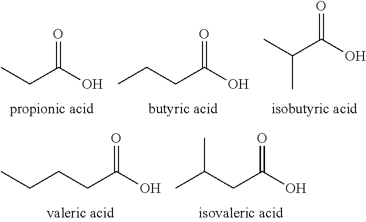

In some embodiments, a compound of the disclosure is pyruvic acid, octanoic acid, dodecanoic acid, (4R)-4-hydroxypentanoic acid, 2-ethylhydracrylic acid, 2-hydroxy-3-methylpentanoic acid, 2-methylbut-2-enoic acid, butanoic acid, methylbutyric acid, dimethylbutyric acid, pentadienoic acid, pentenoic acid, pivalic acid, or propynoic acid. In some embodiments, a compound of the disclosure is pyruvate, octanoate, dodecanoate, (4R)-4-hydroxypentanoate, 2-ethylhydracrylate, 2-hydroxy-3-methylpentanoate, 2-methylbut-2-enoate, butanoate, methylbutyrate, dimethylbutyrate, pentadienoate, pentenoate, pivalate, or propynoate, or a pharmaceutically-acceptable salt of any of the foregoing.

In some embodiments, a compound of the disclosure is an SCFA precursor or derivative thereof. In some embodiments, the compound of the disclosure is lactate, succinate, formate, 1,2-propenediol, tryptamine, indole, indole-3-acetate, or a pharmaceutically-acceptable salt thereof.

In some embodiments, a compound of the disclosure is an SCFA biosynthesis precursor or derivative thereof. In some embodiments, a compound of the disclosure is an acetyl-CoA carboxylase inhibitor, an adenosine monophosphate kinase (AMPK) activator, or vitamin D.

### Pharmaceutically Acceptable Salts

The disclosure provides pharmaceutically-acceptable salts of any therapeutic compound described herein. In some embodiments, the disclosure provides pharmaceutically-acceptable hydrates or solvates of compounds described herein. In some embodiments, the disclosure provides base-addition salts. A base that is added to the compound to form a base-addition salt can be an organic base or an inorganic base. In some embodiments, a pharmaceutically-acceptable salt is a metal salt. In some embodiments, a pharmaceutically-acceptable salt is an ammonium salt.

Metal salts can arise from the addition of an inorganic base to a compound of the disclosure. The inorganic base consists of a metal cation paired with a basic counterion, such as, for example, hydroxide, carbonate, bicarbonate, or phosphate. The metal can be an alkali metal, alkaline earth metal, transition metal, or main group metal. In some embodiments, the metal is lithium, sodium, potassium, cesium, cerium, magnesium, manganese, iron, calcium, strontium, cobalt, titanium, aluminum, copper, cadmium, or zinc.

In some embodiments, a metal salt is a lithium salt, a sodium salt, a potassium salt, a cesium salt, a cerium salt, a magnesium salt, a manganese salt, an iron salt, a calcium salt, a strontium salt, a cobalt salt, a titanium salt, an aluminum salt, a copper salt, a cadmium salt, or a zinc salt.

Ammonium salts can arise from the addition of ammonia or an organic amine to a compound of the disclosure. In some embodiments, the organic amine is triethyl amine, diisopropyl amine, ethanol amine, diethanol amine, triethanol amine, morpholine, N-methylmorpholine, piperidine, N-methylpiperidine, N-ethylpiperidine, dibenzylamine, piperazine, pyridine, pyrrazole, pipyrrazole, imidazole, pyrazine, or pipyrazine.

In some embodiments, an ammonium salt is a triethyl amine salt, a diisopropyl amine salt, an ethanol amine salt, a diethanol amine salt, a triethanol amine salt, a morpholine salt, an N-methylmorpholine salt, a piperidine salt, an N-methylpiperidine salt, an N-ethylpiperidine salt, a dibenzylamine salt, a piperazine salt, a pyridine salt, a pyrrazole salt, a pipyrrazole salt, an imidazole salt, a pyrazine salt, or a pipyrazine salt.

In some embodiments, the salt is a hydrochloride salt, a hydrobromide salt, a hydroiodide salt, a nitrate salt, a nitrite salt, a sulfate salt, a sulfite salt, a phosphate salt, isonicotinate salt, a lactate salt, a salicylate salt, a tartrate salt, an ascorbate salt, a gentisinate salt, a gluconate salt, a glucaronate salt, a saccarate salt, a formate salt, a benzoate salt, a glutamate salt, a pantothenate salt, an acetate salt, a propionate salt, a butyrate salt, a fumarate salt, a succinate salt, a methanesulfonate (mesylate) salt, an ethanesulfonate salt, a benzenesulfonate salt, a p-toluenesulfonate salt, a citrate salt, an oxalate salt, or a maleate salt.

In some embodiments, a compound of the disclosure is an ester of the carboxylic acid. In some embodiments, a compound of the disclosure is an ester of the carboxylic acid with a branched or unbranched alkyl alcohol of 1 to 6 carbon atoms. In some embodiments, a compound of the disclosure can be an ethyl ester, propyl ester, butyl ester, isopropyl ester, t-butyl ester, pentyl ester, or hexyl ester.

### Pharmaceutical Compositions of the Disclosure.

The present disclosure provides pharmaceutical compositions comprising at least one compound of the disclosure. A pharmaceutical composition of the disclosure can be a combination of any compounds described herein with other chemical components, such as carriers, stabilizers, diluents, dispersing agents, suspending agents, thickening agents, and/or excipients. The pharmaceutical composition facilitates administration of the compound to an organism, for example, a subject. Pharmaceutical compositions can be administered in therapeutically-effective amounts as pharmaceutical compositions by various forms and routes including, for example, intravenous, subcutaneous, intramuscular, oral, parenteral, ophthalmic, subcutaneous, transdermal, nasal, vaginal, and topical administration.

A pharmaceutical composition can be administered in a local manner, for example, via injection of the compound directly into an organ, optionally in a depot or sustained release formulation or implant.

In some embodiments, a pharmaceutical composition of the disclosure is formulated for oral administration. In some embodiments, a pharmaceutical composition can be formulated by combining compounds of the disclosure with pharmaceutically-acceptable carriers or excipients. Such carriers can be used to formulate liquids, gels, syrups, elixirs, slurries, or suspensions, for oral ingestion by a subject. Non-limiting examples of solvents used in an oral dissolvable formulation can include water, ethanol, isopropanol, saline, physiological saline, DMSO, dimethylformamide, potassium phosphate buffer, phosphate buffer saline (PBS), sodium phosphate buffer, 4-2-hydroxyethyl-1-piperazineethanesulfonic acid buffer (HEPES), 3-(N-morpholino)propanesulfonic acid buffer (MOPS), piperazine-N,N′-bis(2-ethanesulfonic acid) buffer (PIPES), and saline sodium citrate buffer (SSC). Non-limiting examples of co-solvents used in an oral dissolvable formulation can include sucrose, urea, cremaphor, DMSO, and potassium phosphate buffer.

In some embodiments, pharmaceutical preparations of the disclosure can be formulated for intravenous administration. The pharmaceutical compositions can be in a form suitable for parenteral injection as a sterile suspension, solution or emulsion in oily or aqueous vehicles, and can contain formulatory agents such as suspending, stabilizing and/or dispersing agents.

Pharmaceutical formulations for parenteral administration include aqueous solutions of the active compounds in water-soluble form. Suspensions of the active compounds can be prepared as oily injection suspensions. Suitable lipophilic solvents or vehicles include fatty oils such as sesame oil, or synthetic fatty acid esters, such as ethyl oleate or triglycerides, or liposomes. The suspension can also contain suitable stabilizers or agents which increase the solubility of the compounds to allow for the preparation of highly concentrated solutions. Alternatively, the active ingredient can be in powder form for constitution with a suitable vehicle, e.g., sterile pyrogen-free water, before use.

The active compounds can be administered topically and can be formulated into a variety of topically administrable compositions, such as solutions, suspensions, lotions, gels, pastes, medicated sticks, balms, creams, and ointments. Such pharmaceutical compositions can contain solubilizers, stabilizers, tonicity enhancing agents, buffers and preservatives. The compounds of the disclosure can be applied topically to the skin, or a body cavity, for example, oral, vaginal, bladder, cranial, spinal, thoracic, or pelvic cavity of a subject. The compounds of the disclosure can be applied to an accessible body cavity.

Pharmaceutical compositions can be formulated using one or more physiologically-acceptable carriers comprising excipients and auxiliaries, which facilitate processing of the active compounds into preparations that can be used pharmaceutically. Formulations can be modified depending upon the route of administration chosen. Pharmaceutical compositions comprising a compound described herein can be manufactured, for example, by mixing, dissolving, emulsifying, encapsulating, entrapping, or compression processes.

The pharmaceutical compositions can include at least one pharmaceutically-acceptable carrier, diluent, or excipient and compounds described herein as free-base or pharmaceutically-acceptable salt form. Pharmaceutical compositions can contain solubilizers, stabilizers, tonicity enhancing agents, buffers and preservatives.

Methods for the preparation of compositions comprising the compounds described herein include formulating the compounds with one or more inert, pharmaceutically-acceptable excipients or carriers to form a solid, semi-solid, or liquid composition. Solid compositions include, for example, powders, tablets, dispersible granules, capsules, and cachets. Liquid compositions include, for example, solutions in which a compound is dissolved, emulsions comprising a compound, or a solution containing liposomes, micelles, or nanoparticles comprising a compound as disclosed herein. Semi-solid compositions include, for example, gels, suspensions and creams. The compositions can be in liquid solutions or suspensions, solid forms suitable for solution or suspension in a liquid prior to use, or as emulsions. These compositions can also contain minor amounts of nontoxic, auxiliary substances, such as wetting or emulsifying agents, pH buffering agents, and other pharmaceutically-acceptable additives.

Non-limiting examples of dosage forms suitable for use in the disclosure include liquid, powder, gel, nanosuspension, nanoparticle, microgel, aqueous or oily suspensions, emulsion, and any combination thereof.

Non-limiting examples of pharmaceutically-acceptable excipients suitable for use in the disclosure include binding agents, disintegrating agents, anti-adherents, anti-static agents, surfactants, anti-oxidants, coating agents, coloring agents, plasticizers, preservatives, suspending agents, emulsifying agents, anti-microbial agents, spheronization agents, and any combination thereof. In some embodiments, the pharmaceutically-acceptable excipients of the disclosure are pharmaceutical grade excipients.

Pharmaceutical compositions can be provided in the form of a rapid release formulation, in the form of an extended release formulation, or in the form of an intermediate release formulation. A rapid release form can provide an immediate release. An extended release formulation can provide a controlled release or a sustained delayed release. A pharmaceutical composition of the disclosure can be, for example, an immediate release form or a controlled release formulation. An immediate release formulation can be formulated to allow the compounds to act rapidly. Non-limiting examples of immediate release formulations include readily dissolvable formulations. A controlled release formulation can be a pharmaceutical formulation that has been adapted such that release rates and release profiles of the active agent can be matched to physiological and chronotherapeutic requirements or, alternatively, has been formulated to effect release of an active agent at a programmed rate. Non-limiting examples of controlled release formulations include granules, delayed release granules, hydrogels (e.g., of synthetic or natural origin), other gelling agents (e.g., gel-forming dietary fibers), matrix-based formulations (e.g., formulations comprising a polymeric material having at least one active ingredient dispersed through), granules within a matrix, polymeric mixtures, and granular masses.

In some, a controlled release formulation is a delayed release form. A delayed release form can be formulated to delay a compound's action for an extended period of time. A delayed release form can be formulated to delay the release of an effective dose of one or more compounds, for example, for about 4, about 8, about 12, about 16, or about 24 hours.

A controlled release formulation can be a sustained release form. A sustained release form can be formulated to sustain, for example, the compound's action over an extended period of time. A sustained release form can be formulated to provide an effective dose of any compound described herein (e.g., provide a physiologically-effective blood profile) over about 4, about 8, about 12, about 16 or about 24 hours.

An enteric coating is a polymer barrier applied on oral medication that prevents dissolution or disintegration in the gastric environment. Enteric coatings can protect drugs from the acidity of the stomach, the stomach from detrimental effects of the drug, or to release the drug after the stomach. In some embodiments, the pharmaceutical compositions of the disclosure are provided with an enteric coating. In some embodiments, the enteric coatings of the disclosure are pharmaceutical grade enteric coatings. In some embodiments, the pharmaceutical compositions of the disclosure are provided with an enteric coating that dissolves in the lower gastrointestinal track.

In some embodiments, a material used to provide an enteric coating to a compound of the disclosure is a fatty acid, wax, shellac, plastic, plant fiber, or a film resin. In some embodiments, the enteric coating material is methyl acrylate-methacrylate acid copolymers, cellulose acetate phthalate (CAP), cellulose acetate succinate, hydroxypropyl methyl cellulose phthalate, hydroxypropyl methyl cellulose acetate succinate, hypromellose acetate succinate, polyvinyl acetate phthalate (PVAP), methyl methacrylate-methacrylic acid copolymers, shellac, cellulose acetate trimellitate, sodium alginate, or zein. In some embodiments, the compound is provided as an enteric-coated soft gel, wherein the enteric coating is provided as an enteric coating aqueous solution. In some embodiments, the enteric coating aqueous solution is ethylcellulose, medium chain triglycerides, oleic acid, sodium alginate, or stearic acid.

In some embodiments, the enteric coating is provided as a Vcaps® enteric capsule. In some embodiments, the enteric coating is cellulose acetate phthalate, cellulose acetate trimellitate (CAT), polyvinyl acetate phthalate, hydroxypropylmethylcellulose phthalate, hydroxypropylmethylcellulose acetate succinate, poly (1:1 methacrylic acid:ethyl acrylate), poly (1:1 methacrylic acid:methyl methacrylate), or poly (1:2 methyacrylic acid:methyl methacrylate). In some embodiments, the enteric coating is Eudragit® L30D, Eudragit® L100-55, HP-F, Sureteric®, Acryl-Eze®, Aquarius' Control ENA, Aquateric™, Aquacoat® ECD, or Aquasolve™.

The enteric coating used to coat a pharmaceutical composition of the disclosure can have a thickness of from about 0.5 μm to about 500 μm. In some embodiments, the enteric coating can have a thickness of from about 0.5 μm to about 5 μm, about 5 μm to about 20 μm, about 20 μm to about 50 μm, about 50 μm to about 100 μm, about 100 μm to about 200 μm, about 200 μm to about 300 μm, about 300 μm to about 400 μm, or about 400 μm to about 500 μm. In some embodiments, the enteric coating can have a thickness of about 0.5 μm, about 10 μm, about 25 μm, about 50 μm, about 75 μm, about 100 μm, about 150 μm, about 200 μm, about 250 μm, about 300 μm, about 350 μm, about 400 μm, about 450 μm, or about 500 μm. In some embodiments, the enteric coating has a thickness of about 200 μm. In some embodiments, the enteric coating has a thickness of about 350 μm. In some embodiments, the enteric coating has a thickness of about 500 μm.

Depending on the intended mode of administration, the pharmaceutical compositions can be in the form of solid, semi-solid or liquid dosage forms, such as, for example, tablets, suppositories, pills, capsules, powders, liquids, elixir, suspensions, lotions, creams, or gels, for example, in unit dosage form suitable for single administration of a precise dosage. In some embodiments, a pharmaceutical composition can be in the form of a nanosuspension, aqueous suspension, or oily suspension. In some embodiments, a pharmaceutical composition of the disclosure can be in the form of a drop or syrup.

For solid compositions, nontoxic solid carriers include, for example, pharmaceutical grades of mannitol, lactose, starch, magnesium stearate, sodium saccharin, talc, cellulose, glucose, sucrose, and magnesium carbonate.

Non-limiting examples of pharmaceutically-acceptable excipients suitable for use in the disclosure include granulating agents, binding agents, lubricating agents, disintegrating agents, sweetening agents, glidants, anti-adherents, anti-static agents, surfactants, anti-oxidants, gums, coating agents, coloring agents, flavoring agents, coating agents, plasticizers, preservatives, suspending agents, emulsifying agents, plant cellulosic material and spheronization agents, and any combination thereof. In some embodiments, pharmaceutically-acceptable excipients suitable for use in the disclosure also include adjuvants, anti-oxidants, chelating agents, surfactants, foaming agents, wetting agents, emulsifying agents, viscosifiers, buffering agents, or preservatives.

In some embodiments, the pharmaceutically-acceptable excipient is a permeation enhancer. In some embodiments, the permeation enhancer is ethanol, glycerol monolaurage, polyethylene glycol monolaurate, or dimethylsulfoxide. In some embodiments, the permeation enhancer is oleic acid, oleyl alcohol, ethoxydiglycol, laurocapram, alkanecarboxylic acids, dimethylsulfoxide, polar lipids, or N-methyl-2-pyrrolidone.

In some embodiments, the pharmaceutically-acceptable excipient is a hydrotropic agent. In some embodiments, the hydrotropic agent is isopropyl alcohol, propylene glycol, or sodium xylene sulfonate.

In some embodiments, the pharmaceutically-acceptable excipient is a tablet binder, tablet disintegrant, viscosity increasing agent, tablet or capsule diluent, tablet or capsule disintegrant, thermal stabilizer, adsorbent, film-forming agent, granulating agent, coating agent, flavoring fixative, coloring agent, sweetening agent, or tonicity agent.

In some embodiments, the pharmaceutically-acceptable excipient is acacia, alginate, alginic acid, aluminum acetate, benzyl alcohol, butyl paraben, butylated hydroxy toluene, citric acid, calcium carbonate, candelilla wax, croscarmellose sodium, confectioner sugar, colloidal silicone dioxide, cellulose, plain or anhydrous calcium phosphate, carnauba wax, corn starch, caboxymethylcellulose calcium, calcium stearate, calcium disodium EDTA, copolyvidone, hydrogenated castor oil, calcium hydrogen phosphate dihydrate, cetylpyridine chloride, cysteine HCl, crosspovidone, calcium phosphate di or tri basic, dibasic calcium phosphate, disodium hydrogen phosphate, dimethyicone, erythrosine sodium, ethyl cellulose, gelatin, glyceryl monooleate, glycerine, glycine, glyceryl monostearate, glyceryl behenate, hydroxypropyl cellulose, hydroxy propyl methyl cellulose, Hypromellose, HPMC phthalate, iron oxides, iron oxide yellow, iron oxide red, lactose (hydrous or anhydrous), magnesium stearate, microcrystalline cellulose, mannitol, methyl cellulose, magnesium carbonate, mineral oil, methacrylic acid copolymer, magnesium oxide, methyl paraben, povidone (PVP), polyethylene glycol (PEG), polysorbate 80, propylene glycol, polyethylene oxide, propylene paraben, polaxamer (407, 188, or plain), potassium bicarbonate, potassium sorbate, potato starch, phosphoric acid, polyoxy 140 stearate, sodium starch glycolate, pregelatinized starch, sodium crossmellose, sodium lauryl sulfate, starch, silicone dioxide, sodium benzoate, stearic acid, sucrose, sorbic acid, sodium carbonate, saccharin sodium, sodium alginate, silica gel, sorbiton monooleate, sodium stearyl fumarate, sodium chloride, sodium metabisulfite, sodium citrate dihydrate, sodium starch, sodium carboxymethyl cellulose, succinic acid, sodium propionate, titanium dioxide, talc, triacetin, or triethyl citrate.

Non-limiting examples of pharmaceutically-acceptable excipients can be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), each of which is incorporated by reference in its entirety.

In practicing the methods of treatment or use provided herein, therapeutically-effective amounts of the compounds described herein are administered in pharmaceutical compositions to a subject having a disease or condition to be treated. In some embodiments, the subject is a mammal such as a human. In some embodiments, the subject is an adult, elderly adult, adolescent, pre-adolescent, child, toddler, infant, neonate, or a non-human children, toddlers, infants, neonates, and non-human animals. In some embodiments, a subject is a patient.

Non-limiting examples of pharmaceutically active agents suitable for combination with compositions of the disclosure include anti-infectives, i.e., aminoglycosides, antiviral agents, antimicrobials, anticholinergics/antispasmotics, antidiabetic agents, antihypertensive agents, antineoplastics, cardiovascular agents, central nervous system agents, coagulation modifiers, hormones, immunologic agents, immunosuppressive agents, and ophthalmic preparations.

In some embodiments, a pharmaceutical composition of the disclosure comprises: a) a first short chain fatty acid or a pharmaceutically-acceptable salt thereof; and b) a second short chain fatty acid or a pharmaceutically-acceptable salt thereof, wherein the first short chain fatty acid or the pharmaceutically-acceptable salt thereof and the second short chain fatty acid or the pharmaceutically-acceptable salt thereof are present in the formulation in a ratio of at least 2:1, at least 3:1, at least 4:1, at least 5:1, at least 6:1, at least 7:1, at least 8:1, at least 9:1, at least 10:1, at least 11:1, at least 12:1, at least 13:1, at least 14:1, at least 15:1, at least 16:1, at least 17:1, at least 18:1, at least 19:1, or at least 20:1. In some embodiments, the first short chain fatty acid or the pharmaceutically-acceptable salt thereof and the second short chain fatty acid or the pharmaceutically-acceptable salt thereof are present in the formulation in a ratio of at least 4:1. In some embodiments, the first short chain fatty acid or the pharmaceutically-acceptable salt thereof and the second short chain fatty acid or the pharmaceutically-acceptable salt thereof are present in the formulation in a ratio of at least 10:1. In some embodiments, the first short chain fatty acid or the pharmaceutically-acceptable salt thereof and the second short chain fatty acid or the pharmaceutically-acceptable salt thereof are present in the formulation in a ratio of at least 15:1.

In some embodiments, a pharmaceutical composition of the disclosure comprises: a) a first short chain fatty acid or a pharmaceutically-acceptable salt thereof; and b) a second short chain fatty acid or a pharmaceutically-acceptable salt thereof, wherein the first short chain fatty acid or the pharmaceutically-acceptable salt thereof and the second short chain fatty acid or the pharmaceutically-acceptable salt thereof are present in the formulation in a ratio of from about 2:1 to about 4:1, from about 4:1 to about 6:1, from about 6:1 to about 8:1, from about 8:1 to about 10:1, from about 10:1 to about 12:1, from about 12:1 to about 14:1, from about 14:1 to about 16:1, from about 16:1 to about 18:1, or from about 18:1 to about 20:1. In some embodiments, the first short chain fatty acid or the pharmaceutically-acceptable salt thereof and the second short chain fatty acid or the pharmaceutically-acceptable salt thereof are present in the formulation in a ratio of from about 14:1 to about 16:1.

In some embodiments, a pharmaceutical composition of the disclosure comprises: a) a first short chain fatty acid or a pharmaceutically-acceptable salt thereof; and b) a second short chain fatty acid or a pharmaceutically-acceptable salt thereof, wherein the first short chain fatty acid or the pharmaceutically-acceptable salt thereof and the second short chain fatty acid or the pharmaceutically-acceptable salt thereof are present in the formulation in a ratio of about 2:1, about 3:1, about 4:1, about 5:1, about 6:1, about 7:1, about 8:1, about 9:1, about about 11:1, about 12:1, about 13:1, about 14:1, about 15:1, about 16:1, about 17:1, about 18:1, about 19:1, or about 20:1. In some embodiments, the first short chain fatty acid or the pharmaceutically-acceptable salt thereof and the second short chain fatty acid or the pharmaceutically-acceptable salt thereof are present in the formulation in a ratio of about 10:1. In some embodiments, the first short chain fatty acid or the pharmaceutically-acceptable salt thereof and the second short chain fatty acid or the pharmaceutically-acceptable salt thereof are present in the formulation in a ratio of about 15:1.

In some embodiments, the first short chain fatty acid or the pharmaceutically-acceptable salt thereof is butyrate or a pharmaceutically-acceptable salt thereof. In some embodiments, the first short chain fatty acid or the pharmaceutically-acceptable salt thereof is sodium butyrate. In some embodiments, the first short chain fatty acid is butyric acid. In some embodiments, the second short chain fatty acid or the pharmaceutically-acceptable salt thereof is propionate or a pharmaceutically-acceptable salt thereof. In some embodiments, the second short chain fatty acid is sodium propionate. In some embodiments, the second short chain fatty acid is propionic acid. In some embodiments, the pharmaceutical composition further comprises a pharmaceutically-acceptable excipient. In some embodiments, the pharmaceutically-acceptable excipient is cellulose. In some embodiments, the pharmaceutically-acceptable excipient is methylcellulose. In some embodiments, the pharmaceutically-acceptable excipient is hydroxypropyl cellulose. In some embodiments, the pharmaceutical composition further comprises an enteric coating. In some embodiments, the enteric coating is a Vcaps® enteric capsule. In some embodiments, the enteric coating is CAT. In some embodiments, the pharmaceutical composition is formulated as a tablet. In some embodiments, the pharmaceutical composition is formulated as a capsule.

In some embodiments, a pharmaceutical composition of the disclosure comprises: a) butyrate or a pharmaceutically-acceptable salt thereof; and b) propionate or a pharmaceutically-acceptable salt thereof, wherein butyrate or a pharmaceutically-acceptable salt thereof and propionate or a pharmaceutically-acceptable salt thereof are present in the pharmaceutical composition in a ratio of at least 4:1, at least 5:1, at least 6:1, at least 7:1, at least 8:1, at least 9:1, at least 10:1, at least 11:1, at least 12:1, at least 13:1, at least 14:1, at least 15:1, at least 16:1, at least 17:1, at least 18:1, at least 19:1, or at least 20:1. In some embodiments, a pharmaceutical composition of the disclosure comprises: a) sodium butyrate; and b) sodium propionate; wherein the sodium butyrate and sodium propionate are present in the pharmaceutical composition in a ratio of at least 4:1, at least 5:1, at least 6:1, at least 7:1, at least 8:1, at least 9:1, at least 10:1, at least 11:1, at least 12:1, at least 13:1, at least 14:1, at least 15:1, at least 16:1, at least 17:1, at least 18:1, at least 19:1, or at least 20:1. In some embodiments, the first short chain fatty acid or the pharmaceutically-acceptable salt thereof and the second short chain fatty acid or the pharmaceutically-acceptable salt thereof are present in the formulation in a ratio of at least 4:1. In some embodiments, the first short chain fatty acid or the pharmaceutically-acceptable salt thereof and the second short chain fatty acid or the pharmaceutically-acceptable salt thereof are present in the formulation in a ratio of at least 10:1. In some embodiments, the first short chain fatty acid or the pharmaceutically-acceptable salt thereof and the second short chain fatty acid or the pharmaceutically-acceptable salt thereof are present in the formulation in a ratio of at least 15:1.

In some embodiments, a pharmaceutical composition of the disclosure comprises: a) butyrate or a pharmaceutically-acceptable salt thereof; and b) propionate or a pharmaceutically-acceptable salt thereof, wherein butyrate or a pharmaceutically-acceptable salt thereof and propionate or a pharmaceutically-acceptable salt thereof are present in the pharmaceutical composition in a ratio of about 4:1, about 5:1, about 6:1, about 7:1, about 8:1, about 9:1, about 10:1, about 11:1, about 12:1, about 13:1, about 14:1, about 15:1, about 16:1, about 17:1, about 18:1, about 19:1, or about 20:1. In some embodiments, a pharmaceutical composition of the disclosure comprises: a) sodium butyrate; and b) sodium propionate; wherein the sodium butyrate and sodium propionate are present in the pharmaceutical composition in a ratio of about 4:1, about 5:1, about 6:1, about 7:1, about 8:1, about 9:1, about 10:1, about 11:1, about 12:1, about 13:1, about 14:1, about 15:1, about 16:1, about 17:1, about 18:1, about 19:1, or about 20:1. In some embodiments, the first short chain fatty acid or the pharmaceutically-acceptable salt thereof and the second short chain fatty acid or the pharmaceutically-acceptable salt thereof are present in the formulation in a ratio of about 4:1. In some embodiments, the first short chain fatty acid or the pharmaceutically-acceptable salt thereof and the second short chain fatty acid or the pharmaceutically-acceptable salt thereof are present in the formulation in a ratio of about 10:1. In some embodiments, the first short chain fatty acid or the pharmaceutically-acceptable salt thereof and the second short chain fatty acid or the pharmaceutically-acceptable salt thereof are present in the formulation in a ratio of about 15:1.

In some embodiments, the first short chain fatty acid is butyrate or a pharmaceutically-acceptable salt thereof. In some embodiments, the first short chain fatty acid is butyric acid or a pharmaceutically-acceptable salt thereof. In some embodiments, the first short chain fatty acid is sodium butyrate. In some embodiments, the second short chain fatty acid is propionate or a pharmaceutically-acceptable salt thereof. In some embodiments, the second short chain fatty acid is propionic acid. In some embodiments, the second short chain fatty acid is sodium propionate. In some embodiments, the pharmaceutical composition further comprises a pharmaceutically-acceptable excipient. In some embodiments, the pharmaceutically-acceptable excipient is cellulose. In some embodiments, the pharmaceutically-acceptable excipient is methylcellulose. In some embodiments, the pharmaceutically-acceptable excipient is hydroxypropyl cellulose. In some embodiments, the pharmaceutical composition further comprises an enteric coating. In some embodiments, the enteric coating is a Vcaps® enteric capsule. In some embodiments, the enteric coating is CAT. In some embodiments, the pharmaceutical composition is formulated as a tablet. In some embodiments, the pharmaceutical composition is formulated as a capsule.

### Dosing.

Pharmaceutical compositions described herein can be in unit dosage forms suitable for single administration of precise dosages. In unit dosage form, the formulation is divided into unit doses containing appropriate quantities of one or more pharmaceutical compositions or formulations. The unit dosage can be in the form of a package containing discrete quantities of the pharmaceutical composition or formulation.

In some embodiments, a pharmaceutical composition or formulation of the disclosure is provided as a liquid in a vial or ampoule. In some embodiments, a pharmaceutical composition or formulation of the disclosure is provided as an aqueous suspension packaged in a single-dose non-reclosable container. In some embodiments, a pharmaceutical composition or formulation of the disclosure is provided as an aqueous suspension packaged in a multi-dose reclosable container. Multiple-dose reclosable containers can be used, for example, in combination with a preservative.

In some embodiments, a pharmaceutical composition or formulation of the disclosure is provided as a powder in a single-dose container, for example, a sachet. In some embodiments, a pharmaceutical composition or formulation of the disclosure is provided as a powder in a multi-dose reclosable container. In some embodiments, a pharmaceutical composition or formulation of the disclosure is provided in the form of a tablet. In some embodiments, a pharmaceutical composition or formulation of the disclosure is provided in the form of a capsule.

In some embodiments, a compound described herein can be present in a composition in range of from about 50 mg to about 100 mg, about 100 mg to about 200 mg, about 200 mg to about 300 mg, about 300 mg to about 400 mg, or about 400 mg to about 500 mg. In some embodiments, a compound described herein can be present in a composition in a range of from about 500 mg to about 5,000 mg, from about 1,000 mg to about 5,000 mg, from about 1,500 mg to about 4,000 mg, from about 2,000 mg to about 3,000 mg, or from about 2,500 mg to about 3,000 mg. In some embodiments, a compound described herein can be present in a composition in a range of from about 1,000 mg to about 1,200 mg, from about 1,200 mg to about 1,400 mg, from about 1,400 mg to about 1,600 mg, from about 1,600 mg to about 1,800 mg, from about 1,800 mg to about 2,000 mg, from about 2,000 mg to about 2,200 mg, from about 2,200 mg to about 2,400 mg, from about 2,400 mg to about 2,600 mg, from about 2,600 mg to about 2,800 mg, from about 2,800 mg to about 3,000 mg, from about 3,000 mg to about 3,200 mg, from about 3,200 mg to about 3,400 mg, from about 3,400 mg to about 3,600 mg, from about 3,600 mg to about 3,800 mg, from about 3,800 mg to about 4,000 mg, from about 4,000 mg to about 4,200 mg, from about 4,200 mg to about 4,400 mg, from about 4,400 mg to about 4,600 mg, from about 4,600 mg to about 4,800 mg, or from about 4,800 mg to about 5,000 mg.

In some embodiments, a compound described herein can be present in a composition in an amount of about 50 mg, 100 mg, 150 mg, 200 mg, 250 mg, 300 mg, 350 mg, 400 mg, 450 mg, or 500 mg. In some embodiments, a compound described herein can be present in a composition in an amount of about 500 mg, about 750 mg, about 1,000 mg, about 1,250 mg, about 1,500 mg, about 1,750 mg, about 2,000 mg, about 2,250 mg, about 2,500 mg, about 3,000 mg, about 3,250 mg, about 3,500 mg, about 3,750 mg, about 4,000 mg, about 4,250 mg, about 4,500 mg, about 4,750 mg, or about 5,000 mg.

In some embodiments, a dose can be expressed in terms of an amount of the drug divided by the mass of the subject, for example, milligrams of drug per kilograms of subject body mass. In some embodiments, a compound is administered in an amount ranging from about 1 mg/kg to about 5 mg/kg, about 5 mg/kg to about 10 mg/kg, about 10 mg/kg to about mg/kg, about 15 mg/kg to about 20 mg/kg, about 20 mg/kg to about 25 mg/kg, about 25 mg/kg to about 30 mg/kg, about 30 mg/kg to about 35 mg/kg, about 35 mg/kg to about 40 mg/kg, about 40 mg/kg to about 45 mg/kg, or about 45 mg/kg to about 50 mg/kg, about 50 mg/kg to about 55 mg/kg, about 55 mg/kg to about 60 mg/kg, about 60 mg/kg to about 65 mg/kg, about 65 mg/kg to about 70 mg/kg, or about 70 mg/kg to about 75 mg/kg. In some embodiments, a compound is administered in an amount of about 10 mg/kg, 15 mg/kg, 20 mg/kg, 25 mg/kg, 30 mg/kg, 35 mg/kg, 40 mg/kg, 45 mg/kg, 50 mg/kg, 55 mg/kg, 60 mg/kg, mg/kg, 70 mg/kg, or 75 mg/kg.

In some embodiments, a compound is administered in amount of about 1 mg/kg, about 2 mg/kg, about 3 mg/kg, about 4 mg/kg, about 5 mg/kg, or about 6 mg/kg. In some embodiments, a compound is administered in an amount of about 4 mg/kg. In some embodiments, a compound dis administered in an amount of about 5 mg/kg.

In some embodiments, a compound is administered in an amount of about 50 mg/kg, about 55 mg/kg, about 60 mg/kg, about 65 mg/kg, or about 70 mg/kg. In some embodiments, a compound is administered in an amount of about 65 mg/kg. In some embodiments, a compound is administered in an amount of about 70 mg/kg.

In some embodiments, a pharmaceutical composition comprises 1, 2, 3, 4, or 5 compounds of the disclosure. In some embodiments, a pharmaceutical composition comprises 1 compound of the disclosure. In some embodiments, a pharmaceutical composition comprises 2 compounds of the disclosure. In some embodiments, a pharmaceutical composition comprises 3 compounds of the disclosure.

In some embodiments, a pharmaceutical composition comprises a first compound of the disclosure and a second compound of the disclosure. In some embodiments, a pharmaceutical composition comprises a first compound in an amount of about 150 mg to about 200 mg; and a second compound in an amount of about 3,000 mg to about 3,500 mg. In some embodiments, a pharmaceutical composition comprises a first compound in an amount of about 200 mg to about 250 mg; and a second compound in an amount of about 3,500 mg to about 4,000 mg. In some embodiments, a pharmaceutical composition comprises a first compound in an amount of about 250 mg to about 300 mg; and a second compound in an amount of about 4,000 mg to about 4,500 mg. In some embodiments, a pharmaceutical composition comprises a first compound in an amount of about 250 mg; and a second compound in an amount of about 4,000 mg.

In some embodiments, a pharmaceutical composition comprises a first compound in an amount of about 1 mg/kg to about 5 mg/kg; and a second compound in an amount of about mg/kg to about 70 mg/kg. In some embodiments, a pharmaceutical composition comprises a first compound in an amount of about 4 mg/kg and a second compound in an amount of about 65 mg/kg. In some embodiments, a pharmaceutical composition comprises a first compound in an amount of about 5 mg/kg and a second compound in an amount of 70 mg/kg.

In some embodiments, a pharmaceutical composition comprises butyrate or a pharmaceutically-acceptable salt thereof and one additional SCFA. In some embodiments, a pharmaceutical composition comprises propionate or a pharmaceutically-acceptable salt thereof and butyrate or a pharmaceutically-acceptable salt thereof. In some embodiments, a pharmaceutical composition comprises isobutyrate or a pharmaceutically-acceptable salt thereof and butyrate and a pharmaceutically-acceptable salt thereof. In some embodiments, a pharmaceutical composition comprises butyrate or a pharmaceutically-acceptable salt thereof and valerate or a pharmaceutically-acceptable salt thereof. In some embodiments, a pharmaceutical composition comprises butyrate or a pharmaceutically-acceptable salt thereof and isovalerate or a pharmaceutically-acceptable salt thereof.

In some embodiments, a pharmaceutical composition comprises sodium butyrate and one additional SCFA. In some embodiments, a pharmaceutical composition comprises sodium propionate and sodium butyrate. In some embodiments, a pharmaceutical composition comprises sodium isobutyrate and sodium butyrate. In some embodiments, a pharmaceutical composition comprises sodium butyrate and sodium valerate. In some embodiments, a pharmaceutical composition comprises sodium butyrate and sodium isovalerate.

In some embodiments, a pharmaceutical composition comprises butyric acid and one additional SCFA. In some embodiments, a pharmaceutical composition comprises propionic acid and butyric acid. In some embodiments, a pharmaceutical composition comprises isobutyric acid and butyric acid. In some embodiments, a pharmaceutical composition comprises butyric acid and valeric acid. In some embodiments, a pharmaceutical composition comprises butyric acid and isovaleric acid.

In some embodiments, a pharmaceutical composition comprises 250 mg propionate or a pharmaceutically-acceptable salt thereof and 4,000 mg butyrate or a pharmaceutically-acceptable salt thereof. In some embodiments, a pharmaceutical composition comprises 250 mg isobutyrate or a pharmaceutically-acceptable salt thereof and 4,000 mg butyrate or a pharmaceutically-acceptable salt thereof. In some embodiments, a pharmaceutical composition comprises 250 mg valerate or a pharmaceutically-acceptable salt thereof and 4,000 mg butyrate or a pharmaceutically-acceptable salt thereof. In some embodiments, a pharmaceutical composition comprises 250 mg isovalerate or a pharmaceutically-acceptable salt thereof and 4,000 mg butyrate or a pharmaceutically-acceptable salt thereof.

In some embodiments, a pharmaceutical composition comprises 250 mg sodium propionate and 4,000 mg sodium butyrate. In some embodiments, a pharmaceutical composition comprises 250 mg sodium isobutyrate and 4,000 mg sodium butyrate. In some embodiments, a pharmaceutical composition comprises 250 mg sodium valerate and 4,000 mg sodium butyrate. In some embodiments, a pharmaceutical composition comprises 250 mg sodium isovalerate and 4,000 mg sodium butyrate.

In some embodiments, a pharmaceutical composition comprises 250 mg propionic acid and 4,000 mg butyric acid. In some embodiments, a pharmaceutical composition comprises 250 mg isobutyric acid and 4,000 mg butyric acid. In some embodiments, a pharmaceutical composition comprises 250 mg valeric acid and 4,000 mg butyric acid. In some embodiments, a pharmaceutical composition comprises 250 mg isovaleric acid and 4,000 mg butyric acid.

In some embodiments, a pharmaceutical composition comprises isobutyrate or a pharmaceutically-acceptable salt thereof and one additional SCFA. In some embodiments, a pharmaceutical composition comprises isobutyrate or a pharmaceutically-acceptable salt thereof and propionate or a pharmaceutically-acceptable salt thereof. In some embodiments, a pharmaceutical composition comprises isobutyrate or a pharmaceutically-acceptable salt thereof and valerate or a pharmaceutically-acceptable salt thereof. In some embodiments, a pharmaceutical composition comprises isobutyrate or a pharmaceutically-acceptable salt thereof and isovalerate or a pharmaceutically-acceptable salt thereof.

In some embodiments, a pharmaceutical composition comprises sodium isobutyrate and one additional SCFA. In some embodiments, a pharmaceutical composition comprises sodium isobutyrate and sodium propionate. In some embodiments, a pharmaceutical composition comprises sodium isobutyrate and sodium valerate. In some embodiments, a pharmaceutical composition comprises sodium isobutyrate and sodium isovalerate.

In some embodiments, a pharmaceutical composition comprises isobutyric acid and one additional SCFA. In some embodiments, a pharmaceutical composition comprises isobutyric acid and propionic acid. In some embodiments, a pharmaceutical composition comprises isobutyric acid and valeric acid. In some embodiments, a pharmaceutical composition comprises isobutyric acid and isovaleric acid.

In some embodiments, a pharmaceutical composition comprises 250 mg propionate or a pharmaceutically-acceptable salt thereof and 4,000 mg isobutyrate or a pharmaceutically-acceptable salt thereof. In some embodiments, a pharmaceutical composition comprises 250 mg valerate or a pharmaceutically-acceptable salt thereof and 4,000 mg isobutyrate or a pharmaceutically-acceptable salt thereof. In some embodiments, a pharmaceutical composition comprises 250 mg isovalerate or a pharmaceutically-acceptable salt thereof and 4,000 mg isobutyrate or a pharmaceutically-acceptable salt thereof.

In some embodiments, a pharmaceutical composition comprises 250 mg sodium propionate and 4,000 mg sodium isobutyrate. In some embodiments, a pharmaceutical composition comprises 250 mg sodium valerate and 4,000 mg sodium isobutyrate. In some embodiments, a pharmaceutical composition comprises 250 mg sodium isovalerate and 4,000 mg sodium isobutyrate.

In some embodiments, a pharmaceutical composition comprises 250 mg propionic acid and 4,000 mg isobutyric acid. In some embodiments, a pharmaceutical composition comprises 250 mg valeric acid and 4,000 mg isobutyric acid. In some embodiments, a pharmaceutical composition comprises 250 mg isovaleric acid and 4,000 mg isobutyric acid.

### Methods of Administration

Pharmaceutical compositions or therapeutic agents described herein can be administered before, during, or after the occurrence of a disease or condition, and the timing of administering the composition containing a therapeutic agent can vary. For example, the pharmaceutical compositions or therapeutic agents can be used as a prophylactic and can be administered continuously to subjects with a propensity to conditions or diseases in order to lessen a likelihood of the occurrence of the disease or condition. The pharmaceutical compositions or therapeutic agents can be administered to a subject during or as soon as possible after the onset of the symptoms. The administration of the pharmaceutical compositions or therapeutic agents can be initiated within the first 48 hours of the onset of the symptoms, within the first 24 hours of the onset of the symptoms, within the first 6 hours of the onset of the symptoms, or within 3 hours of the onset of the symptoms. The initial administration can be via any route practical, such as by any route described herein using any formulation described herein.

In some embodiments, pharmaceutical compositions or therapeutic agents can be administered to a patient exhibiting early symptoms of an illness. In some embodiments, the symptom is coughing. In some embodiments, the symptom is fever. Pharmaceutical compositions or therapeutic agents can be administered as soon as is practical after the onset of a disease or condition is detected or suspected, and for a length of time necessary for the treatment of the disease, such as, for example, from about 1 month to about 3 months. In some embodiments, the length of time pharmaceutical compositions or therapeutic agents can be administered can be about 1 day, about 2 days, about 3 days, about 4 days, about 5 days, about 6 days, about 1 week, about 2 weeks, about 3 weeks, about 4 weeks, about 1 month, about 5 weeks, about 6 weeks, about 7 weeks, about 8 weeks, about 2 months, about 9 weeks, about 10 weeks, about 11 weeks, about 12 weeks, about 3 months, about 13 weeks, about 14 weeks, about 15 weeks, about 16 weeks, about 4 months, about 17 weeks, about 18 weeks, about 19 weeks, about 20 weeks, about 5 months, about 21 weeks, about 22 weeks, about 23 weeks, about 24 weeks, about 6 months, about 7 months, about 8 months, about 9 months, about 10 months, about 11 months, about 1 year, about 13 months, about 14 months, about 15 months, about 16 months, about 17 months, about 18 months, about 19 months, about 20 months, about 21 months, about 22 months about 23 months, about 2 years, about 2.5 years, about 3 years, about 3.5 years, about 4 years, about 4.5 years, about 5 years, about 6 years, about 7 years, about 8 years, about 9 years, or about 10 years. The length of treatment can vary for each subject.

Multiple pharmaceutical compositions or therapeutic agents can be administered in any order or simultaneously. In some embodiments, a pharmaceutical composition of the disclosure is administered in combination with, before, or after treatment with another therapeutic agent. If simultaneously, the pharmaceutical compositions or therapeutic agents can be provided in a single, unified form, or in multiple forms, for example, as multiple separate pills. The pharmaceutical compositions or therapeutic agents can be packed together or separately, in a single package or in a plurality of packages. One or all of the pharmaceutical composition or therapeutic agents can be given in multiple doses. If not simultaneous, the timing between the multiple doses can vary to as much as about a month.

In some embodiments, a pharmaceutical composition is administered in a daily oral dose. In some embodiments, a pharmaceutical composition is administered in a daily oral dose 3 times a day. In some embodiments, a formulation is administered in a daily oral dose 3 times a day for one week. In some embodiments, a formulation is administered in a daily oral dose 3 times a day for two weeks. In some embodiments, a formulation is administered in a daily oral dose 3 times a day for three weeks.

In some embodiments, a pharmaceutical composition is administered in a daily oral dose with food. In some embodiments, a pharmaceutical composition is administered in a daily oral dose 3 times a day, each time with food. In some embodiments, a formulation is administered in a daily oral dose 3 times a day for one week, each time with food. In some embodiments, a formulation is administered in a daily oral dose 3 times a day for two weeks, each time with food. In some embodiments, a formulation is administered in a daily oral dose 3 times a day for three weeks, each time with food.

In some embodiments, a pharmaceutical composition is administered in a daily oral dose after food. In some embodiments, a pharmaceutical composition is administered in a daily oral dose 3 times a day, each time after food. In some embodiments, a formulation is administered in a daily oral dose 3 times a day for one week, each time after food. In some embodiments, a formulation is administered in a daily oral dose 3 times a day for two weeks, each time after food. In some embodiments, a formulation is administered in a daily oral dose 3 times a day for three weeks, each time after food. In some embodiments, a formulation is administered about 5 minutes, about 15 minutes, about 30 minutes, or about an hour after food.

In some embodiments, a pharmaceutical composition is administered in a daily oral dose. In some embodiments, a pharmaceutical composition is administered in a daily oral dose 3 times a day. In some embodiments, a formulation is administered in a daily oral dose 3 times a day for one month. In some embodiments, a formulation is administered in a daily oral dose 3 times a day for two months. In some embodiments, a formulation is administered in a daily oral dose 3 times a day for three months. In some embodiments, a formulation is administered in a daily oral dose 3 times a day for one year. In some embodiments, a formulation is administered in a daily oral dose 3 times a day for two years. In some embodiments, a formulation is administered in a daily oral dose 3 times a day for three years.

In some embodiments, a pharmaceutical composition is administered in a daily oral dose with food. In some embodiments, a pharmaceutical composition is administered in a daily oral dose 3 times a day, each time with food. In some embodiments, a formulation is administered in a daily oral dose 3 times a day for one month, each time with food. In some embodiments, a formulation is administered in a daily oral dose 3 times a day for two months, each time with food. In some embodiments, a formulation is administered in a daily oral dose 3 times a day for three months, each time with food. In some embodiments, a formulation is administered in a daily oral dose 3 times a day for one year, each time with food. In some embodiments, a formulation is administered in a daily oral dose 3 times a day for two years, each time with food. In some embodiments, a formulation is administered in a daily oral dose 3 times a day for three years, each time with food.

In some embodiments, a pharmaceutical composition is administered in a daily oral dose after food. In some embodiments, a pharmaceutical composition is administered in a daily oral dose 3 times a day, each time after food. In some embodiments, a formulation is administered in a daily oral dose 3 times a day for one month, each time after food. In some embodiments, a formulation is administered in a daily oral dose 3 times a day for two months, each time after food. In some embodiments, a formulation is administered in a daily oral dose 3 times a day for three months, each time after food. In some embodiments, a formulation is administered in a daily oral dose 3 times a day for one year, each time after food. In some embodiments, a formulation is administered in a daily oral dose 3 times a day for two years, each time after food. In some embodiments, a formulation is administered in a daily oral dose 3 times a day for three years, each time after food. In some embodiments, a formulation is administered about 5 minutes, about 15 minutes, about 30 minutes, or about an hour after food.

In some embodiments, a pharmaceutical composition comprising butyrate or a pharmaceutically-acceptable salt thereof and propionate or a pharmaceutically-acceptable salt thereof is administered in a daily oral dose. In some embodiments, a pharmaceutical composition comprising butyrate or a pharmaceutically-acceptable salt thereof and propionate or a pharmaceutically-acceptable salt thereof is administered in a daily oral dose 3 times a day. In some embodiments, a pharmaceutical composition comprising butyrate or a pharmaceutically-acceptable salt thereof and propionate or a pharmaceutically-acceptable salt thereof is administered in a daily oral dose 3 times a day. In some embodiments, a pharmaceutical composition comprising butyrate or a pharmaceutically-acceptable salt thereof and propionate or a pharmaceutically-acceptable salt thereof is administered in a daily oral dose 3 times a day for one week. In some embodiments, a pharmaceutical composition comprising butyrate or a pharmaceutically-acceptable salt thereof and propionate or a pharmaceutically-acceptable salt thereof is administered in a daily oral dose 3 times a day for two weeks. In some embodiments, a pharmaceutical composition comprising butyrate or a pharmaceutically-acceptable salt thereof and propionate or a pharmaceutically-acceptable salt thereof is administered in a daily oral dose 3 times a day for three weeks.

In some embodiments, a pharmaceutical composition comprising butyrate or a pharmaceutically-acceptable salt thereof and propionate or a pharmaceutically-acceptable salt thereof is administered in a daily oral dose with food. In some embodiments, a pharmaceutical composition comprising butyrate or a pharmaceutically-acceptable salt thereof and propionate or a pharmaceutically-acceptable salt thereof is administered in a daily oral dose 3 times a day, each time with food. In some embodiments, a pharmaceutical composition comprising butyrate or a pharmaceutically-acceptable salt thereof and propionate or a pharmaceutically-acceptable salt thereof is administered in a daily oral dose 3 times a day, each time with food. In some embodiments, a pharmaceutical composition comprising butyrate or a pharmaceutically-acceptable salt thereof and propionate or a pharmaceutically-acceptable salt thereof is administered in a daily oral dose 3 times a day for one week, each time with food. In some embodiments, a pharmaceutical composition comprising butyrate or a pharmaceutically-acceptable salt thereof and propionate or a pharmaceutically-acceptable salt thereof is administered in a daily oral dose 3 times a day for two weeks, each time with food. In some embodiments, a pharmaceutical composition comprising butyrate or a pharmaceutically-acceptable salt thereof and propionate or a pharmaceutically-acceptable salt thereof is administered in a daily oral dose 3 times a day for three weeks, each time with food.

In some embodiments, a pharmaceutical composition comprising butyrate or a pharmaceutically-acceptable salt thereof and propionate or a pharmaceutically-acceptable salt thereof is administered in a daily oral dose after food. In some embodiments, a pharmaceutical composition comprising butyrate or a pharmaceutically-acceptable salt thereof and propionate or a pharmaceutically-acceptable salt thereof is administered in a daily oral dose 3 times a day, each time after food. In some embodiments, a pharmaceutical composition comprising butyrate or a pharmaceutically-acceptable salt thereof and propionate or a pharmaceutically-acceptable salt thereof is administered in a daily oral dose 3 times a day, each time after food. In some embodiments, a pharmaceutical composition comprising butyrate or a pharmaceutically-acceptable salt thereof and propionate or a pharmaceutically-acceptable salt thereof is administered in a daily oral dose 3 times a day for one week, each time after food. In some embodiments, a pharmaceutical composition comprising butyrate or a pharmaceutically-acceptable salt thereof and propionate or a pharmaceutically-acceptable salt thereof is administered in a daily oral dose 3 times a day for two weeks, each time after food. In some embodiments, a pharmaceutical composition comprising butyrate or a pharmaceutically-acceptable salt thereof and propionate or a pharmaceutically-acceptable salt thereof is administered in a daily oral dose 3 times a day for three weeks, each time after food. In some embodiments, a formulation is administered about 5 minutes, about 15 minutes, about 30 minutes, or about an hour after food.

In some embodiments, a pharmaceutical composition comprising sodium butyrate and sodium propionate is administered in a daily oral dose. In some embodiments, a pharmaceutical composition comprising sodium butyrate and sodium propionate is administered in a daily oral dose 3 times a day. In some embodiments, a pharmaceutical composition comprising sodium butyrate and sodium propionate is administered in a daily oral dose 3 times a day for one week. In some embodiments, a pharmaceutical composition comprising sodium butyrate and sodium propionate is administered in a daily oral dose 3 times a day for two weeks. In some embodiments, a pharmaceutical composition comprising sodium butyrate and sodium propionate is administered in a daily oral dose 3 times a day for three weeks.

In some embodiments, a pharmaceutical composition comprising sodium butyrate and sodium propionate is administered in a daily oral dose with food. In some embodiments, a pharmaceutical composition comprising sodium butyrate and sodium propionate is administered in a daily oral dose 3 times a day, each time with food. In some embodiments, a pharmaceutical composition comprising sodium butyrate and sodium propionate is administered in a daily oral dose 3 times a day for one week, each time with food. In some embodiments, a pharmaceutical composition comprising sodium butyrate and sodium propionate is administered in a daily oral dose 3 times a day for two weeks, each time with food. In some embodiments, a pharmaceutical composition comprising sodium butyrate and sodium propionate is administered in a daily oral dose 3 times a day for three weeks, each time with food.

In some embodiments, a pharmaceutical composition comprising sodium butyrate and sodium propionate is administered in a daily oral dose after food. In some embodiments, a pharmaceutical composition comprising sodium butyrate and sodium propionate is administered in a daily oral dose 3 times a day, each time after food. In some embodiments, a pharmaceutical composition comprising sodium butyrate and sodium propionate is administered in a daily oral dose 3 times a day for one week, each time after food. In some embodiments, a pharmaceutical composition comprising sodium butyrate and sodium propionate is administered in a daily oral dose 3 times a day for two weeks, each time after food. In some embodiments, a pharmaceutical composition comprising sodium butyrate and sodium propionate is administered in a daily oral dose 3 times a day for three weeks, each time after food. In some embodiments, a formulation is administered about 5 minutes, about 15 minutes, about 30 minutes, or about an hour after food.

In some embodiments, a pharmaceutical composition comprising butyric acid and propionic acid, or pharmaceutically-acceptable salts thereof, is administered in a daily oral dose. In some embodiments, a pharmaceutical composition comprising butyric acid and propionic acid, or a pharmaceutically-acceptable salt of each, is administered in a daily oral dose 3 times a day. In some embodiments, a pharmaceutical composition comprising butyric acid and propionic acid, or pharmaceutically-acceptable salts thereof, is administered in a daily oral dose 3 times a day. In some embodiments, a pharmaceutical composition comprising butyric acid and propionic acid, or pharmaceutically-acceptable salts thereof, is administered in a daily oral dose 3 times a day for one week. In some embodiments, a pharmaceutical composition comprising butyric acid and propionic acid, or pharmaceutically-acceptable salts thereof, is administered in a daily oral dose 3 times a day for two weeks. In some embodiments, a pharmaceutical composition comprising butyric acid and propionic acid, or pharmaceutically-acceptable salts thereof, is administered in a daily oral dose 3 times a day for three weeks.

In some embodiments, a pharmaceutical composition comprising butyric acid and propionic acid, or pharmaceutically-acceptable salts thereof, is administered in a daily oral dose with food. In some embodiments, a pharmaceutical composition comprising butyric acid and propionic acid, or a pharmaceutically-acceptable salt of each, is administered in a daily oral dose 3 times a day, each time with food. In some embodiments, a pharmaceutical composition comprising butyric acid and propionic acid, or pharmaceutically-acceptable salts thereof, is administered in a daily oral dose 3 times a day, each time with food. In some embodiments, a pharmaceutical composition comprising butyric acid and propionic acid, or pharmaceutically-acceptable salts thereof, is administered in a daily oral dose 3 times a day for one week, each time with food. In some embodiments, a pharmaceutical composition comprising butyric acid and propionic acid, or pharmaceutically-acceptable salts thereof, is administered in a daily oral dose 3 times a day for two weeks, each time with food. In some embodiments, a pharmaceutical composition comprising butyric acid and propionic acid, or pharmaceutically-acceptable salts thereof, is administered in a daily oral dose 3 times a day for three weeks, each time with food.

In some embodiments, a pharmaceutical composition comprising butyric acid and propionic acid, or pharmaceutically-acceptable salts thereof, is administered in a daily oral dose after food. In some embodiments, a pharmaceutical composition comprising butyric acid and propionic acid, or a pharmaceutically-acceptable salt of each, is administered in a daily oral dose 3 times a day, each time after food. In some embodiments, a pharmaceutical composition comprising butyric acid and propionic acid, or pharmaceutically-acceptable salts thereof, is administered in a daily oral dose 3 times a day, each time after food. In some embodiments, a pharmaceutical composition comprising butyric acid and propionic acid, or pharmaceutically-acceptable salts thereof, is administered in a daily oral dose 3 times a day for one week, each time after food. In some embodiments, a pharmaceutical composition comprising butyric acid and propionic acid, or pharmaceutically-acceptable salts thereof, is administered in a daily oral dose 3 times a day for two weeks, each time after food. In some embodiments, a pharmaceutical composition comprising butyric acid and propionic acid, or pharmaceutically-acceptable salts thereof, is administered in a daily oral dose 3 times a day for three weeks, each time after food. In some embodiments, a formulation is administered about 5 minutes, about 15 minutes, about 30 minutes, or about an hour after food.

In some embodiments, a pharmaceutical composition is administered in a daily parenteral dose. In some embodiments, a pharmaceutical composition is administered in a daily parenteral dose 3 times a day. In some embodiments, a formulation is administered in a daily parenteral dose 3 times a day for one week. In some embodiments, a formulation is administered in a daily parenteral dose 3 times a day for two weeks. In some embodiments, a formulation is administered in a daily parenteral dose 3 times a day for three weeks.

In some embodiments, a pharmaceutical composition is administered in a daily parenteral dose. In some embodiments, a pharmaceutical composition is administered in a daily parenteral dose 3 times a day. In some embodiments, a formulation is administered in a daily parenteral dose 3 times a day for one month. In some embodiments, a formulation is administered in a daily parenteral dose 3 times a day for two months. In some embodiments, a formulation is administered in a daily parenteral dose 3 times a day for three months. In some embodiments, a formulation is administered in a daily parenteral dose 3 times a day for one year. In some embodiments, a formulation is administered in a daily parenteral dose 3 times a day for two years. In some embodiments, a formulation is administered in a daily parenteral dose 3 times a day for three years.

In some embodiments, a pharmaceutical composition comprising butyrate or a pharmaceutically-acceptable salt thereof and propionate or a pharmaceutically-acceptable salt thereof is administered in a daily parenteral dose. In some embodiments, a pharmaceutical composition comprising butyrate or a pharmaceutically-acceptable salt thereof and propionate or a pharmaceutically-acceptable salt thereof is administered in a daily parenteral dose 3 times a day. In some embodiments, a pharmaceutical composition comprising butyrate or a pharmaceutically-acceptable salt thereof and propionate or a pharmaceutically-acceptable salt thereof is administered in a daily parenteral dose 3 times a day for one week. In some embodiments, a pharmaceutical composition comprising butyrate or a pharmaceutically-acceptable salt thereof and propionate or a pharmaceutically-acceptable salt thereof is administered in a daily parenteral dose 3 times a day for two weeks. In some embodiments, a pharmaceutical composition comprising butyrate or a pharmaceutically-acceptable salt thereof and propionate or a pharmaceutically-acceptable salt thereof is administered in a daily parenteral dose 3 times a day for three weeks.

In some embodiments, a pharmaceutical composition comprising sodium butyrate and sodium propionate is administered in a daily parenteral dose. In some embodiments, a pharmaceutical composition comprising sodium butyrate and sodium propionate is administered in a daily parenteral dose 3 times a day. In some embodiments, a pharmaceutical composition comprising sodium butyrate and sodium propionate is administered in a daily parenteral dose 3 times a day for one week. In some embodiments, a pharmaceutical composition comprising sodium butyrate and sodium propionate is administered in a daily parenteral dose 3 times a day for two weeks. In some embodiments, a pharmaceutical composition comprising sodium butyrate and sodium propionate is administered in a daily parenteral dose 3 times a day for three weeks.

In some embodiments, a pharmaceutical composition comprising butyric acid and propionic acid, or pharmaceutically-acceptable salts thereof, is administered in a daily parenteral dose. In some embodiments, a pharmaceutical composition comprising butyric acid and propionic acid, or pharmaceutically-acceptable salts thereof, is administered in a daily parenteral dose 3 times a day. In some embodiments, a pharmaceutical composition comprising butyric acid and propionic acid, or pharmaceutically-acceptable salts thereof, is administered in a daily parenteral dose 3 times a day for one week. In some embodiments, a pharmaceutical composition comprising butyric acid and propionic acid, or pharmaceutically-acceptable salts thereof, is administered in a daily parenteral dose 3 times a day for two weeks. In some embodiments, a pharmaceutical composition comprising butyric acid and propionic acid, or pharmaceutically-acceptable salts thereof, is administered in a daily parenteral dose 3 times a day for three weeks.

Pharmaceutical compositions described herein can be in unit dosage forms suitable for single administration of precise dosages. In unit dosage form, the formulation is divided into unit doses containing appropriate quantities of one or more compounds. The unit dosage can be in the form of a package containing discrete quantities of the formulation. Non-limiting examples are packaged injectables, vials, or ampoules. Aqueous suspension compositions can be packaged in single-dose non-reclosable containers. Multiple-dose reclosable containers can be used, for example, in combination with or without a preservative. Formulations for injection can be presented in unit dosage form, for example, in ampoules, or in multi-dose containers with a preservative.

A pharmaceutical composition of the disclosure can be used, for example, before, during, or after treatment of a subject with, for example, another pharmaceutical agent. In some embodiments, a pharmaceutical composition of the disclosure is administered with an antiviral agent. In some embodiments, a pharmaceutical composition of the disclosure is administered with an antibiotic agent. Pharmaceutical compositions provided herein, can be administered in conjunction with other therapies, for example, chemotherapy, radiation, surgery, anti-inflammatory agents, and selected vitamins. The other agents can be administered prior to, after, or concomitantly with the pharmaceutical compositions.

A therapeutically-effective amount can vary widely depending on the severity of the disease, the age and relative health of the subject, the potency of the compounds used, and other factors. The compounds can be used singly or in combination with one or more therapeutic agents as components of mixtures. In some embodiments, the compounds can be used in combination with 1, 2, 3, 4, 5, 6, 7, 8, 9, or 10 additional therapeutic agents. In some embodiments, the compounds of the disclosure can be used with 1 additional therapeutic agent. In some embodiments, the compounds of the disclosure can be used with 2 additional therapeutic agents. In some embodiments, the compounds of the disclosure can be used with 3 additional therapeutic agents. In some embodiments, a pharmaceutical composition of the disclosure is administered with an antiviral agent. In some embodiments, a pharmaceutical composition of the disclosure is administered with an antibiotic agent.

### Diseases.

CRS is associated with a wide variety of infectious and noninfectious diseases. A pharmaceutical composition of the disclosure can be used to treat, ameliorate the effects of, or delay the onset of CRS. In some embodiments, CRS is caused by a viral respiratory infection. In some embodiments, CRS is caused by cytomegalovirus, Epstein-Barr virus-associated hemophagocytic lymphohistiocytosis, group A streptococcus, influenza virus, variola virus, Ebola, or coronavirus. In some embodiments, CRS is caused by graft-versus-host disease (GVHD), acute respiratory distress syndrome (ARDS), sepsis, or systemic inflammatory response syndrome. In some embodiments, CRS is caused by severe acute respiratory syndrome coronavirus (SARS-CoV). In some embodiments, CRS is caused by SARS-CoV-2. In some embodiments, CRS is caused by Middle East respiratory syndrome (MERS-CoV). In some embodiments, CRS is caused by H5N1 influenza (Avian flu) or H1N1 influenza (Swine flu). In some embodiments, CRS is caused by an adenovirus infection or SARS.

Severe acute respiratory syndrome coronavirus 2 (SARS-CoV-2) can progress to a hyperinflammatory condition, often with life-threatening pulmonary involvement. This systemic hyperinflammation results in inflammatory lymphocytic and monocytic infiltration of the lung and the heart, causing ARDS and cardiac failure.

A compound of the disclosure can be used to treat a disease characterized by tissue destruction mediated by a cytokine storm. In some embodiments, a compound of the disclosure is used to treat multiple sclerosis, sepsis, cancer treated with CAR-T immunotherapy, and pancreatitis.

In some embodiments, a pharmaceutical composition of the disclosure is used to treat CRS caused by administering a monoclonal antibody drug. In some embodiments, a pharmaceutical composition of the disclosure is used to treat CRS caused by administration of a CD20 antibody (e.g., rituximab), CD19 CART cells (e.g., tisagenlecleucel), theralizumab, mixed bacterial vaccines (MBVs), an anti-CD3 monoclonal antibody (e.g., muromonab-CD3), or an anti-CD52 monoclinal antibody (e.g., alemtuzumab).

In some embodiments, a pharmaceutical composition of the disclosure is used to treat CRS caused by administering an adoptive T-cell therapy. In some embodiments, a pharmaceutical composition of the disclosure is used to treat CRS caused by adoptive cell transfer of autologous T-cells modified with chimeric antigen receptors (CAR-T cell therapy). In some embodiments, a pharmaceutical composition of the disclosure is used to treat CRS in patients with elevated serum levels of IL-6, IFN-γ, IL-8, IL-10, GM-CSF, MIP-1α/β, MCP-1, CXCL0, or CXCL10.

In some embodiments, the disclosure describes a method of treating a condition comprising: administering to a subject in need thereof a pharmaceutical composition comprising a therapeutically-effective amount of a first short chain fatty acid or a pharmaceutically-acceptable salt thereof and a therapeutically-effective amount of a second short chain fatty acid or a pharmaceutically-acceptable salt thereof, wherein the first short chain fatty acid or the pharmaceutically-acceptable salt thereof and the second short chain fatty acid or the pharmaceutically-acceptable salt thereof are present in the formulation in a ratio of at least 10:1, wherein the administering reduces a level of a cytokine in the subject.

In some embodiments, the condition is systemic inflammatory response syndrome. In some embodiments, the systemic inflammatory response syndrome is cytokine release syndrome. In some embodiments, the systemic inflammatory response syndrome is caused by a severe acute respiratory syndrome corona virus 2 (SARS-CoV-2) infection. In some embodiments, the systemic inflammatory response syndrome is caused by an influenza infection. In some embodiments, the condition is acute respiratory distress syndrome.

In some embodiments, the first short chain fatty acid or the pharmaceutically-acceptable salt thereof and the second short chain fatty acid or the pharmaceutically-acceptable salt thereof are present in the formulation in a ratio of about 10:1. In some embodiments, the first short chain fatty acid or the pharmaceutically-acceptable salt thereof and the second short chain fatty acid or the pharmaceutically-acceptable salt thereof are present in the formulation in a ratio of about 15:1. In some embodiments, the first short chain fatty acid is butyrate or a pharmaceutically-acceptable salt thereof. In some embodiments, the first short chain fatty acid is sodium butyrate. In some embodiments, the first short chain fatty acid is butyric acid. In some embodiments, the therapeutically-effective amount of the first short chain fatty acid is about 4000 mg. In some embodiments, the second short chain fatty acid is propionate or a pharmaceutically-acceptable salt thereof. In some embodiments, the second short chain fatty acid is sodium propionate. In some embodiments, the second short chain fatty acid is propionic acid. In some embodiments, the therapeutically-effective amount of the second short chain fatty acid or the pharmaceutically-acceptable salt thereof is about 250 mg. In some embodiments, the pharmaceutical composition further comprises a pharmaceutically-acceptable excipient.

In some embodiments, the administering reduces a level of interferon in the subject. In some embodiments, the administering reduces a level of interleukin in the subject. In some embodiments, the administering reduces a level of tumor necrosis factor in the subject. In some embodiments, the administering reduces a level of TNF-α in the subject. In some embodiments, the administering reduces a level of plasma TNF-α in the subject.

In some embodiments, the administering reduces a level of TNF-α by at least about 2-fold, at least about 5-fold, at least about 10-fold, at least about 15-fold, at least about 20-fold, at least about 25-fold, at least about 30-fold, at least about 35-fold, at least about 40-fold, at least about 45-fold, at least about 50-fold, at least about 55-fold, at least about 60-fold, at least about 65-fold, at least about 70-fold, at least about 75-fold, or at least about 80-fold. In some embodiments, the administering reduces a level of TNF-α by at least about 10-fold. In some embodiments, the administering reduces a level of TNF-α by at least about 20-fold. In some embodiments, the administering reduces a level of TNF-α by at least about 50-fold. In some embodiments, the administering reduces a level of TNF-α by at least about 60-fold.

In some embodiments, the administering reduces a level of TNF-α by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95%. In some embodiments, the administering reduces a level of TNF-α by at least about 15%. In some embodiments, the administering reduces a level of TNF-α by at least about 20%. In some embodiments, the administering reduces a level of TNF-α by at least about 30%. In some embodiments, the administering reduces a level of TNF-α by at least about 40%. In some embodiments, the administering reduces a level of TNF-α by at least about 50%. In some embodiments, the administering reduces a level of TNF-α by at least about 60%. In some embodiments, the administering reduces a level of TNF-α by at least about 70%. In some embodiments, the administering reduces a level of TNF-α by at least about 80%. In some embodiments, the administering reduces a level of TNF-α by at least about 85%.

In some embodiments, the administering reduces a level of TNF-α for at least about 2 hours, at least about 4 hours, at least about 6 hours, at least about 8 hours, at least about 10 hours, at least about 12 hours, at least about 14 hours, at least about 16 hours, at least about 18 hours, at least about 20 hours, at least about 22 hours, at least about 24 hours, at least about 26 hours, at least about 28 hours, at least about 30 hours, at least about 32 hours, at least about 34 hours, at least about 36 hours, at least about 38 hours, at least about 40 hours, at least about 42 hours, at least about 44 hours, at least about 46 hours, or at least about 48 hours. In some embodiments, the administering reduces a level of TNF-α for at least about 1 day, at least about 2 days, at least about 3 days, at least about 4 days, at least about 5 days, at least about 6 days, at least about 7 days, at least about 8 days, at least about 9 days, at least about 10 days, at least about 11 days, at least about 12 days, at least about 13 days, at least about 14 days, at least about 3 weeks, at least about 4 weeks, at least about 5 weeks, at least about 6 weeks, at least about 7 weeks, at least about 8 weeks, at least about 9 weeks, at least about 10 weeks, at least about 11 weeks, at least about 12 weeks, at least about 13 weeks, at least about 14 weeks, at least about 15 weeks, at least about 16 weeks, at least about 17 weeks, at least about 18 weeks, at least about 19 weeks, or at least about 20 weeks. In some embodiments, the administering reduces a level of TNF-α for at least about 6 hours. In some embodiments, the administering reduces a level of TNF-α for at least about 12 hours. In some embodiments, the administering reduces a level of TNF-α for at least about 18 hours. In some embodiments, the administering reduces a level of TNF-α for at least about 24 hours.

In some embodiments, the method further comprises administering a therapeutically-effective amount of a second therapeutic agent. In some embodiments, the second therapeutic agent is an antiviral agent. In some embodiments, the second therapeutic is an antibiotic.

In some embodiments, the disclosure describes a method comprising: administering to a subject in need thereof a pharmaceutical composition comprising a therapeutically-effective amount of a first short chain fatty acid or a pharmaceutically-acceptable salt thereof and a therapeutically-effective amount of a second short chain fatty acid or a pharmaceutically-acceptable salt thereof, wherein the first short chain fatty acid or the pharmaceutically-acceptable salt thereof and the second short chain fatty acid or the pharmaceutically-acceptable salt thereof are present in the formulation in a ratio of at least 10:1, wherein the administering modulates an effect of the subject's immune system.

In some embodiments, the administering reduces a level of a cytokine in a subject. In some embodiments, the administering reduces a level of interferon in the subject. In some embodiments, the administering reduces a level of interleukin in the subject. In some embodiments, the administering reduces a level of tumor necrosis factor in the subject. In some embodiments, the administering reduces a level of a pro-inflammatory cytokine, for example, IL-6, IL-12, IL-17, or IL-23.

In some embodiments, the administering upregulates a level of a cytokine in a subject. In some embodiments, the administering upregulates an anti-inflammatory cytokine, for example, IL-10. In some embodiments, the administering downmodulates NF-κB. In some embodiments, the administering downmodulates NF-κB by β-arrestin recruitment by FFAR2.

In some embodiments, the first short chain fatty acid or the pharmaceutically-acceptable salt thereof and the second short chain fatty acid or the pharmaceutically-acceptable salt thereof are present in the formulation in a ratio of about 10:1. In some embodiments, the first short chain fatty acid or the pharmaceutically-acceptable salt thereof and the second short chain fatty acid or the pharmaceutically-acceptable salt thereof are present in the formulation in a ratio of about 15:1. In some embodiments, the first short chain fatty acid or the pharmaceutically-acceptable salt thereof is butyrate or a pharmaceutically-acceptable salt thereof. In some embodiments, the first short chain fatty acid is sodium butyrate. In some embodiments, the first short chain fatty acid or the pharmaceutically-acceptable salt thereof is butyric acid. In some embodiments, the therapeutically-effective amount of the first short chain fatty acid or the pharmaceutically-acceptable salt thereof is about 4,000 mg. In some embodiments, the second short chain fatty acid or the pharmaceutically-acceptable salt thereof is propionate or a pharmaceutically-acceptable salt thereof. In some embodiments, the second short chain fatty acid is sodium propionate. In some embodiments, the second short chain fatty acid is propionic acid. In some embodiments, the therapeutically-effective amount of the second short chain fatty acid or the pharmaceutically-acceptable salt thereof is about 250 mg. In some embodiments, the pharmaceutical composition further comprises a pharmaceutically-acceptable excipient.

### Kits

Compositions of the disclosure can be packaged as a kit. In some embodiments, a kit includes written instructions on the administration or use of the composition. The written material can be, for example, a label. The written material can suggest conditional methods of administration. The instructions provide the subject and the supervising physician with the best guidance for achieving the optimal clinical outcome from the administration of the therapy. The written material can be a label. In some embodiments, the label can be approved by a regulatory agency, for example, the U.S. Food and Drug Administration (FDA), the European Medicines Agency (EMA), or other regulatory agencies.

### EXAMPLES

### Example 1: Pharmaceutical Compositions

Formulation 1: A tablet comprising 250 mg propionic acid and 4,000 mg butyric acid is prepared. The tablet is coated with an enteric coating material. 120 tablets are prepared in a reclosable container and provided to the patient.

Formulation 2: A powder comprising 250 mg sodium propionate and 4,000 mg sodium butyrate per unit dose is prepared. One unit dose of the powder is portioned into a Vcaps® enteric capsule. 120 capsules are prepared in a reclosable container and provided to the patient.

### Example 2: Mouse LPS-Induced Model of Pulmonary Inflammation

A SCFA solution of 62.6 mg of sodium butyrate and 12.5 mg of sodium propionate was prepared per 1 mL of PBS. The effect of the solution was evaluated in a lipopolysaccharide (LPS)-induced model of pulmonary inflammation.

Animals: Male CD-1 mice, age 7-8 weeks, were used in the study. The animals were randomly assigned to treatment and placebo groups. The mice were acclimated/conditioned for at least 3 days. The mice were housed on a 12 hr light/dark cycle (lights on at 7:00 am). No more than four mice were kept per cage depending on the size. The cages were a ventilated cage rack system. The mice were given standard rodent chow and water ad libitum.

Experimental design: Routes of administration for the SCFA solution was oral (PO). LPS was administered interperitoneally (IP). The SCFA solution (Test article; TA) was dissolved in PBS buffer and administered 10 mL/kg (PO), 3 times a day for 2 hours. The study duration was 2 days. Pre-treatment time was one hour prior to LPS. There were 3 groups of animals, each group having 8 animals.

TABLE 1 summarizes the experimental design of the study

TABLE 1

| | | | TA | | Treatment time | |
|---|---|---|---|---|---|---|
| | Group | LPS | Dose and | | prior to LPS | Evaluation |
| Treatment | size | dosing | route | | administration | Endpoints |
| LPS/Vehicle | 8 | 250 μg IP | 0 | PO | 1 hr | Blood collection at |
| LPS/TA | | 250 μg IP | 10 | mL/kg PO | | 6 hr and 26 hr (post- |
| Vehicle/Vehicle | | 0 μg IP | 0 | PO | | LPS) (250 μL) |
| | | | | | | IL6 and TNF-α |
| | | | | | | measurements in |
| | | | | | | plasma (48 samples |
| | | | | | | each) |
| | | | | | | Lung collection |
| | | | | | | (frozen) |

LPS procedure: Lipopolysaccharide (heat killed E. coli 0127:B5) were prepared in water at a concentration of 0.025 mg/mL and injected at a volume of 10 mL/kg (IP) for a final dose of 0.25 mg/kg. Compound or vehicle were pre-administered to mice 1 hr before LPS injection. Mice were bled by retro-orbital eye-bleed 6 hr after LPS injection and by cardiac puncture 26 hours after LPS injection. TNF-α and IL-6 serum levels were measured by ELISA.

Compound dosing and blood and tissue sampling: Vehicle and TA were administered 1 hr prior to LPS administration and then TID for 26 hours (post LPS; 3 additional doses). Blood (approx. 250 μL) was collected 6 hr and 26 hr after LPS administration and processed for plasma. TABLE 2 shows the schedule of events for the study. Lungs were collected and snap frozen on dry ice.

TABLE 2

| | |
|---|---|
| Time | Event |

Day 1

| | |
|---|---|
| 8 am | Gavage 1 for TA |
| 9 am | IP administration of LPS/Vehicle |
| 1 pm | Gavage 2 for TA |
| 3 pm | Retro orbital eye bleed |
| 5:30 pm | Gavage 3 for TA |

Day 2

| | |
|---|---|
| 8 am | Gavage 4 for TA |
| 11 am | Takedown with terminal bleed and dissection |

### Results

TABLE 3 shows the results of the study.

TABLE 3

| | | VEH/VEH | VEH/LPS | TA/LPS |
|---|---|---|---|---|
| | | (pg/mL) | (pg/mL) | (pg/mL) |
| IL-6 | 6 hr post LPS | 30.56 | 1198.13 | 1452.79 |
| | 26 hr post LPS | 23.09 | 36.66 | 34.88 |
| TNF-α | 6 hr post LPS | 12.42 | 61.86 | 53.93 |
| | 26 hr post LPS | 2.59 | 3.79 | 0.87 |

Plasma IL-6 levels: Treatment with LPS produced a 39-fold and 47.5-fold increase in plasma IL-6 levels compared to vehicle-treated controls 6 hr and 26 hr post-LPS, respectively. Treatment with the SCFA solution did not produce a significant effect on plasma IL-6 at 6 hr and 26 hr post-LPS.

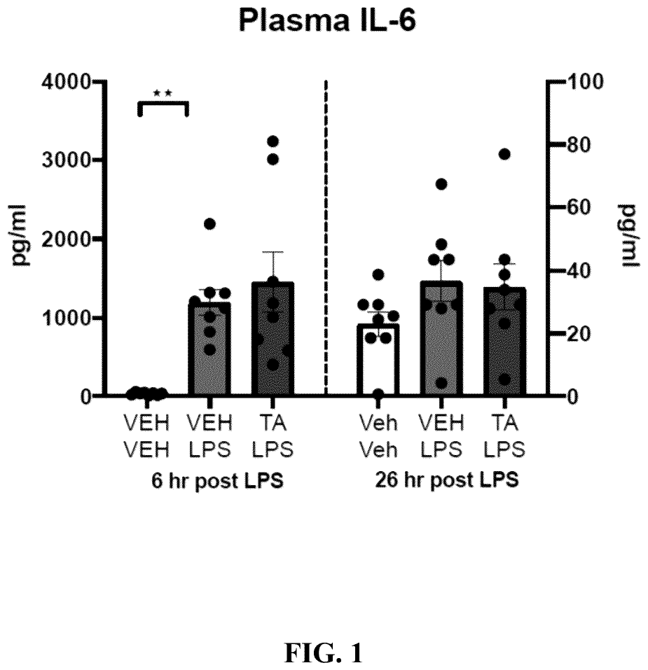

FIG. 1 shows plasma IL-6 levels measured by ELISA. The left section of the graph shows plasma IL-6 levels 6 hours after mice were treated with the vehicle without LPS; vehicle and LPS; and the test article and LPS. The right section of the graph shows plasma IL-6 levels 26 hours after mice were treated with vehicle without LPS; vehicle with LPS; and the test article and LPS. Data are mean±SEM and analyzed by one-way ANOVA using Fisher's uncorrected LSD test. **p<0.01, compared to vehicle/vehicle group. N=8/group/timepoint.

Plasma TNF-α: Treatment with LPS produced a 5-fold and 4.3-fold increase in plasma TNF-α levels compared to vehicle-treated controls 6 hr and 26 hr post-LPS, respectively. Treatment with the SCFA solution did not produce a significant effect on plasma IL-6 at 6 hr post-LPS. A statistically significant effect was observed on plasma TNF-α at 26 hr post-LPS upon treatment with the SCFA solution, with an almost 60-fold decrease in plasma TNF-α levels.

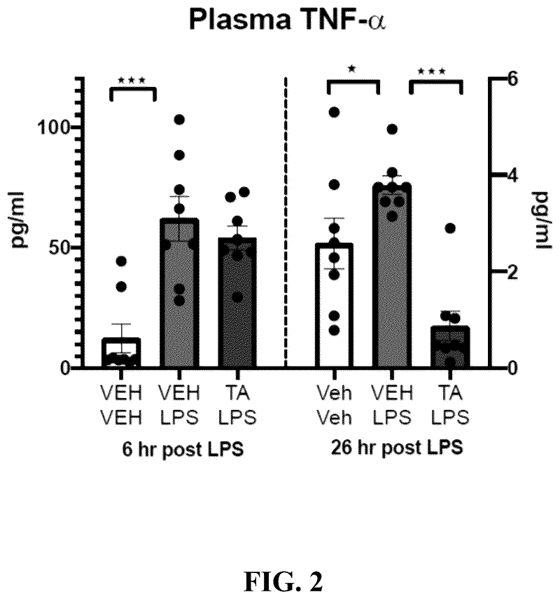

FIG. 2 shows plasma TNF-α measured by ELISA. The left section of the graph shows plasma TNF-α levels 6 hours after mice were treated with the vehicle without LPS; vehicle and LPS; and the test article and LPS. The right section of the graph shows plasma TNF-α levels 26 hours after mice were treated with vehicle without LPS; vehicle with LPS; and the test article and LPS. Data are mean±SEM and analyzed by one-way ANOVA using Fisher's uncorrected LSD test. *p<0.05, ***<0.001 compared to vehicle/vehicle group.

N=8/group/timepoint. The data show that treatment with a SCFA composition comprising butyrate and propionate reduced plasma TNF-α levels in an LPS-induced model of pulmonary inflammation. The reduction in plasma TNF-α increased over time. After about 6 hours of administering the vehicle or TA, the SCFA composition decreased plasma TNF-α levels by about 15% compared to vehicle treatment. After about 26 hours, the SCFA composition decreased plasma TNF-α levels by about 85% compared to vehicle treatment, indicating that the SCFA composition reduced inflammation in the LPS-induced model of pulmonary inflammation.

### Example 3: Clinical Trial for Administration of Sodium Butyrate to Treat COVID-19 Related ARDS

Forty COVID-19 infected patients with mild symptoms of disease (i.e., coughing and fever) are recruited into the clinical trial. The subjects ingest tablets or capsules containing sodium butyrate or a pharmaceutically-acceptable salt thereof and propionate or a pharmaceutically-acceptable salt thereof, described in EXAMPLE 1, 3 times a day right after food. The subject ingests the tablets daily for 2 weeks. Tablets are taken at home.

Sputum and blood samples are obtained from the subjects weekly. Virus levels, clinical symptoms, and pro-inflammatory cytokine levels are measured for each patient. Telemedicine communication is used to assess and guide patients through administration of sodium butyrate and sample collection.

Among patients who do not clear virus during the treatment period, treatment is extended. Clinical symptoms, virus levels, and levels of virus neutralizing antibodies are measured for the extended treatment period.

### EMBODIMENTS

The following non-limiting embodiments provide illustrative examples of the invention but do not limit the scope of the invention.
Embodiment 1. A method of treating a condition comprising: administering to a subject in need thereof a pharmaceutical composition comprising a therapeutically-effective amount of a first short chain fatty acid or a pharmaceutically-acceptable salt thereof and a therapeutically-effective amount of a second short chain fatty acid or a pharmaceutically-acceptable salt thereof, wherein the first short chain fatty acid or the pharmaceutically-acceptable salt thereof and the second short chain fatty acid or the pharmaceutically-acceptable salt thereof are present in the formulation in a ratio of at least 10:1.
Embodiment 2. The method of embodiment 1, wherein the condition is systemic inflammatory response syndrome.
Embodiment 3. The method of embodiment 2, wherein the systemic inflammatory response syndrome is cytokine release syndrome.
Embodiment 4. The method of embodiment 2, wherein the systemic inflammatory response syndrome is caused by a severe acute respiratory syndrome coronavirus 2 (SARS-CoV-2) infection.
Embodiment 5. The method of embodiment 2, wherein the systemic inflammatory response syndrome is caused by an influenza infection.
Embodiment 6. The method of embodiment 1, wherein the condition is acute respiratory distress syndrome.
Embodiment 7. The method of any one of embodiments 1-6, wherein the first short chain fatty acid or the pharmaceutically-acceptable salt thereof and the second short chain fatty acid or the pharmaceutically-acceptable salt thereof are present in the formulation in a ratio of about 10:1.
Embodiment 8. The method of any one of embodiments 1-6, wherein the first short chain fatty acid or the pharmaceutically-acceptable salt thereof and the second short chain fatty acid or the pharmaceutically-acceptable salt thereof are present in the formulation in a ratio of about 15:1.
Embodiment 9. The method of any one of embodiments 1-8, wherein the first short chain fatty acid or the pharmaceutically-acceptable salt thereof is butyrate or a pharmaceutically-acceptable salt thereof
Embodiment 10. The method of any one of embodiments 1-9, wherein the therapeutically-effective amount of the first short chain fatty acid or the pharmaceutically-acceptable salt thereof is about 4,000 mg.
Embodiment 11. The method of any one of embodiments 1-10, wherein the second short chain fatty acid or the pharmaceutically-acceptable salt thereof is propionate or a pharmaceutically-acceptable salt thereof
Embodiment 12. The method of any one of embodiments 1-11, wherein the therapeutically-effective amount of the second short chain fatty acid or the pharmaceutically-acceptable salt thereof is about 250 mg.
Embodiment 13. The method of any one of embodiments 1-12, wherein the pharmaceutical composition further comprises a pharmaceutically-acceptable excipient.
Embodiment 14. The method of embodiment 13, wherein the pharmaceutically-acceptable excipient is cellulose.
Embodiment 15. The method of any one of embodiments 1-14, wherein the administering modulates an effect of the subject's immune system.
Embodiment 16. The method of any one of embodiments 1-15, wherein the administering reduces a level of a cytokine in the subject.
Embodiment 17. The method of embodiment 16, wherein the cytokine is an interferon.
Embodiment 18. The method of embodiment 16, wherein the cytokine is an interleukin.
Embodiment 19. The method of embodiment 16, wherein the cytokine is a tumor necrosis factor.
Embodiment 20. The method of any one of embodiments 1-19, further comprising administering a therapeutically-effective amount of a second therapeutic agent.
Embodiment 21. The method of embodiment 20, wherein the second therapeutic agent is an antiviral agent.
Embodiment 22. The method of embodiment 20, wherein the second therapeutic agent is an antibiotic.
Embodiment 23. A method of treating a condition comprising: administering to a subject in need thereof a pharmaceutical composition comprising a therapeutically-effective amount of a first short chain fatty acid or a pharmaceutically-acceptable salt thereof and a therapeutically-effective amount of a second short chain fatty acid or a pharmaceutically-acceptable salt thereof, wherein the administering reduces a level of a cytokine in the subject.
Embodiment 24. The method of embodiment 23, wherein the condition is systemic inflammatory response syndrome.
Embodiment 25. The method of embodiment 24, wherein the systemic inflammatory response syndrome is cytokine release syndrome.
Embodiment 26. The method of embodiment 24, wherein the systemic inflammatory response syndrome is caused by a severe acute respiratory syndrome coronavirus 2 (SARS-CoV-2) infection.
Embodiment 27. The method of embodiment 24, wherein the systemic inflammatory response syndrome is caused by an influenza infection.
Embodiment 28. The method of embodiment 23, wherein the condition is acute respiratory distress syndrome.
Embodiment 29. The method of any one of embodiments 23-28, wherein the first short chain fatty acid or the pharmaceutically-acceptable salt thereof and the second short chain fatty acid or the pharmaceutically-acceptable salt thereof are present in the formulation in a ratio of at least 4:1.
Embodiment 30. The method of any one of embodiments 23-29, wherein the first short chain fatty acid or the pharmaceutically-acceptable salt thereof and the second short chain fatty acid or the pharmaceutically-acceptable salt thereof are present in the formulation in a ratio of about 4:1.
Embodiment 31. The method of any one of embodiments 23-29, wherein the first short chain fatty acid or the pharmaceutically-acceptable salt thereof and the second short chain fatty acid or the pharmaceutically-acceptable salt thereof are present in the formulation in a ratio of about 10:1.
Embodiment 32. The method of any one of embodiments 23-29, wherein the first short chain fatty acid or the pharmaceutically-acceptable salt thereof and the second short chain fatty acid or the pharmaceutically-acceptable salt thereof are present in the formulation in a ratio of about 15:1.
Embodiment 33. The method of any one of embodiments 23-32, wherein the first short chain fatty acid or the pharmaceutically-acceptable salt thereof is butyrate or a pharmaceutically-acceptable salt thereof
Embodiment 34. The method of any one of embodiments 23-33, wherein the therapeutically-effective amount of the first short chain fatty acid or the pharmaceutically-acceptable salt thereof is about 4,000 mg.
Embodiment 35. The method of any one of embodiments 23-34, wherein the second short chain fatty acid or the pharmaceutically-acceptable salt thereof is propionate or a pharmaceutically-acceptable salt thereof
Embodiment 36. The method of any one of embodiments 23-35, wherein the therapeutically-effective amount is about 250 mg.
Embodiment 37. The method of any one of embodiments 23-36, wherein the pharmaceutical composition further comprises a pharmaceutically-acceptable excipient.
Embodiment 38. The method of embodiment 37, wherein the pharmaceutically-acceptable excipient is cellulose.
Embodiment 39. The method of any one of embodiments 23-38, wherein the cytokine is an interferon.
Embodiment 40. The method of any one of embodiments 23-38, wherein the cytokine is an interleukin.
Embodiment 41. The method of any one of embodiments 23-38, wherein the cytokine is a tumor necrosis factor.
Embodiment 42. The method of any one of embodiments 23-41, further comprising administering a therapeutically-effective amount of a second therapeutic agent.
Embodiment 43. The method of embodiment 42, wherein the second therapeutic agent is an antiviral agent.
Embodiment 44. The method of embodiment 42, wherein the second therapeutic agent is an antibiotic.
Embodiment 45. A method of treating a condition comprising: administering to a subject in need thereof a pharmaceutical composition comprising a therapeutically-effective amount of a first short chain fatty acid or a pharmaceutically-acceptable salt thereof and a therapeutically-effective amount of a second short chain fatty acid or a pharmaceutically-acceptable salt thereof, wherein the administering modulates an effect of the subject's immune system.
Embodiment 46. The method of embodiment 45, wherein the condition is systemic inflammatory response syndrome.
Embodiment 47. The method of embodiment 46, wherein the systemic inflammatory response syndrome is cytokine release syndrome.
Embodiment 48. The method of embodiment 46, wherein the systemic inflammatory response syndrome is caused by a severe acute respiratory syndrome coronavirus 2 (SARS-CoV-2) infection.
Embodiment 49. The method of embodiment 46, wherein the systemic inflammatory response syndrome is caused by an influenza infection.
Embodiment 50. The method of embodiment 45, wherein the condition is acute respiratory distress syndrome.
Embodiment 51. The method of any one of embodiments 45-50, wherein the first short chain fatty acid or the pharmaceutically-acceptable salt thereof and the second short chain fatty acid or the pharmaceutically-acceptable salt thereof are present in the formulation in a ratio of at least 4:1.
Embodiment 52. The method of any one of embodiments 45-50, wherein the first short chain fatty acid or the pharmaceutically-acceptable salt thereof and the second short chain fatty acid or the pharmaceutically-acceptable salt thereof are present in the formulation in a ratio of about 4:1.
Embodiment 53. The method of any one of embodiments 45-50, wherein the first short chain fatty acid or the pharmaceutically-acceptable salt thereof and the second short chain fatty acid or the pharmaceutically-acceptable salt thereof are present in the formulation in a ratio of about 10:1.
Embodiment 54. The method of any one of embodiments 45-50, wherein the first short chain fatty acid or the pharmaceutically-acceptable salt thereof and the second short chain fatty acid or the pharmaceutically-acceptable salt thereof are present in the formulation in a ratio of about 15:1.
Embodiment 55. The method of any one of embodiments 45-54, wherein the first short chain fatty acid or the pharmaceutically-acceptable salt thereof is butyrate or a pharmaceutically-acceptable salt thereof
Embodiment 56. The method of any one of embodiments 45-55, wherein the therapeutically-effective amount of the first short chain fatty acid or the pharmaceutically-acceptable salt thereof is about 4,000 mg.
Embodiment 57. The method of any one of embodiments 45-56, wherein the second short chain fatty acid or the pharmaceutically-acceptable salt thereof is propionate or a pharmaceutically-acceptable salt thereof
Embodiment 58. The method of any one of embodiments 45-57, wherein the therapeutically-effective amount of the second short chain fatty acid or the pharmaceutically-acceptable salt thereof is about 250 mg.
Embodiment 59. The method of any one of embodiments 45-58, wherein the pharmaceutical composition further comprises a pharmaceutically-acceptable excipient.
Embodiment 60. The method of embodiment 45, wherein the pharmaceutically-acceptable excipient is cellulose.
Embodiment 61. The method of any one of embodiments 45-60, wherein the administering reduces a level of a cytokine in the subject.
Embodiment 62. The method of embodiment 61, wherein the cytokine is an interferon.
Embodiment 63. The method of embodiment 61, wherein the cytokine is an interleukin.
Embodiment 64. The method of embodiment 61, wherein the cytokine is a tumor necrosis factor.
Embodiment 65. The method of any one of embodiments 45-64, further comprising administering a therapeutically-effective amount of a second therapeutic agent.
Embodiment 66. The method of embodiment 65, wherein the second therapeutic agent is an antiviral agent.
Embodiment 67. The method of embodiment 65, wherein the second therapeutic agent is an antibiotic.
Embodiment 68. A pharmaceutical composition comprising: a) a first short chain fatty acid or a pharmaceutically-acceptable salt thereof; and b) a second short chain fatty acid or a pharmaceutically-acceptable salt thereof, wherein the first short chain fatty acid or the pharmaceutically-acceptable salt thereof and the second short chain fatty acid or the pharmaceutically-acceptable salt thereof are present in the formulation in a ratio of at least 10:1.
Embodiment 69. The pharmaceutical composition of embodiment 68, wherein the first short chain fatty acid or the pharmaceutically-acceptable salt thereof and the second short chain fatty acid or the pharmaceutically-acceptable salt thereof are present in the formulation in a ratio of about 10:1.
Embodiment 70. The pharmaceutical composition of embodiment 68, wherein the first short chain fatty acid or the pharmaceutically-acceptable salt thereof and the second short chain fatty acid or the pharmaceutically-acceptable salt thereof are present in the formulation in a ratio of about 15:1.
Embodiment 71. The pharmaceutical composition of any one of embodiments 68-70, wherein the first short chain fatty acid or the pharmaceutically-acceptable salt thereof is butyrate or a pharmaceutically-acceptable salt thereof
Embodiment 72. The pharmaceutical composition of embodiment 71, wherein the pharmaceutical composition comprises about 4000 mg of butyrate or the pharmaceutically-acceptable salt thereof
Embodiment 73. The pharmaceutical composition of any one of embodiments 68-72, wherein the second short chain fatty acid or the pharmaceutically-acceptable salt thereof is propionate or the pharmaceutically-acceptable salt thereof
Embodiment 74. The pharmaceutical composition of embodiment 73, wherein the pharmaceutical composition comprises about 250 mg of propionate or the pharmaceutically-acceptable salt thereof
Embodiment 75. The pharmaceutical composition of any one of embodiments 68-74, wherein the pharmaceutical composition further comprises a pharmaceutically-acceptable excipient.
Embodiment 76. The pharmaceutical composition of embodiment 75, wherein the pharmaceutically-acceptable excipient is cellulose.
Embodiment 77. The pharmaceutical composition of any one of embodiments 68-76, wherein the pharmaceutical composition further comprises an enteric coating.
Embodiment 78. The pharmaceutical composition of embodiment 77, wherein the enteric coating is a Vcaps® capsule.
Embodiment 79. The pharmaceutical composition of any one of embodiments 68-78, wherein the pharmaceutical composition is formulated as a tablet.
Embodiment 80. The pharmaceutical composition of any one of embodiments 68-78, wherein the pharmaceutical composition is formulated as a capsule.
Embodiment 81. A pharmaceutical composition comprising: a) butyrate or a pharmaceutically-acceptable salt thereof; and b) propionate or a pharmaceutically-acceptable salt thereof; wherein butyrate or the pharmaceutically-acceptable salt thereof and propionate or the pharmaceutically-acceptable salt thereof are present in the pharmaceutical composition in a ratio of at least 10:1.
Embodiment 82. The pharmaceutical composition of embodiment 81, wherein the butyrate or the pharmaceutically-acceptable salt thereof and the propionate or the pharmaceutically-acceptable salt thereof are present in the formulation in a ratio of about
Embodiment 83. The pharmaceutical composition of embodiment 81, wherein the butyrate or the pharmaceutically-acceptable salt thereof and the propionate or the pharmaceutically-acceptable salt thereof are present in the formulation in a ratio of about 15:1.
Embodiment 84. The pharmaceutical composition of any one of embodiments 81-83, wherein the butyrate or the pharmaceutically-acceptable salt thereof is present in an amount of about 3000 mg to about 5000 mg.
Embodiment 85. The pharmaceutical composition of any one of embodiments 81-84, wherein the butyrate or the pharmaceutically-acceptable salt thereof is present in an amount of about 4000 mg.
Embodiment 86. The pharmaceutical composition of any one of embodiments 81-85, wherein the propionate or the pharmaceutically-acceptable salt thereof is present in an amount of about 100 mg to about 500 mg.
Embodiment 87. The pharmaceutical composition of any one of embodiments 81-86, wherein the propionate or the pharmaceutically-acceptable salt thereof is present in an amount of about 250 mg.
Embodiment 88. The pharmaceutical composition of any one of embodiments 81-87, wherein the pharmaceutical composition further comprises a pharmaceutically-acceptable excipient.
Embodiment 89. The pharmaceutical composition of embodiment 84, wherein the pharmaceutically-acceptable excipient is a cellulose.
Embodiment 90. The pharmaceutical composition of embodiment 89, wherein the cellulose is methylcellulose.
Embodiment 91. The pharmaceutical composition of embodiment 90, wherein the methylcellulose is hydroxypropyl methylcellulose.
Embodiment 92. The pharmaceutical composition of any one of embodiments 81-91, wherein the pharmaceutical composition further comprises an enteric coating.
Embodiment 93. The pharmaceutical composition of embodiment 92, wherein the enteric coating is a Vcaps® capsule.
Embodiment 94. The pharmaceutical composition of any one of embodiments 81-93, further comprising at least one pharmaceutically-acceptable excipient.
Embodiment 95. The pharmaceutical composition of any one of embodiments 81-94, wherein the pharmaceutical composition is formulated as a tablet.
Embodiment 96. The pharmaceutical composition of any one of embodiments 81-94, wherein the pharmaceutical composition is formulated as a capsule.

What is claimed is:

1. A method of treating a condition comprising:
administering to a subject in need thereof a pharmaceutical composition comprising a therapeutically-effective amount of a first short chain fatty acid or a pharmaceutically-acceptable salt thereof and a therapeutically-effective amount of a second short chain fatty acid or a pharmaceutically-acceptable salt thereof,
wherein the first short chain fatty acid or the pharmaceutically-acceptable salt thereof and the second short chain fatty acid or the pharmaceutically-acceptable salt thereof are present in the formulation in a ratio of at least 10:1.

2. The method of claim 1, wherein the condition is systemic inflammatory response syndrome.

3. The method of claim 2, wherein the systemic inflammatory response syndrome is cytokine release syndrome.

4. The method of claim 2, wherein the systemic inflammatory response syndrome is caused by a severe acute respiratory syndrome coronavirus 2 (SARS-CoV-2) infection.

5. The method of claim 2, wherein the systemic inflammatory response syndrome is caused by an influenza infection.

6. The method of claim 1, wherein the condition is acute respiratory distress syndrome.

7. The method of claim 1, wherein the first short chain fatty acid or the pharmaceutically-acceptable salt thereof and the second short chain fatty acid or the pharmaceutically-acceptable salt thereof are present in the formulation in a ratio of about 10:1.

8. The method of claim 1, wherein the first short chain fatty acid or the pharmaceutically-acceptable salt thereof and the second short chain fatty acid or the pharmaceutically-acceptable salt thereof are present in the formulation in a ratio of about 15:1.

9. The method of claim 1, wherein the first short chain fatty acid or the pharmaceutically-acceptable salt thereof is butyrate or a pharmaceutically-acceptable salt thereof.

10. The method of claim 9, wherein the therapeutically-effective amount of the first short chain fatty acid or the pharmaceutically-acceptable salt thereof is about 4,000 mg.

11. The method of claim 1, wherein the second short chain fatty acid or the pharmaceutically-acceptable salt thereof is propionate or a pharmaceutically-acceptable salt thereof.

12. The method of claim 11, wherein the therapeutically-effective amount of the second short chain fatty acid is about 250 mg.

13. The method of claim 1, wherein the pharmaceutical composition further comprises a pharmaceutically-acceptable excipient.

14. The method of claim 13, wherein the pharmaceutically-acceptable excipient is cellulose.

15. The method of claim 1, wherein the administering modulates an effect of the subject's immune system.

16. The method of claim 15, wherein the administering reduces a level of a cytokine in the subject.

17. The method of claim 16, wherein the cytokine is an interferon.

18. The method of claim 16, wherein the cytokine is an interleukin.

19. The method of claim 16, wherein the cytokine is a tumor necrosis factor.

20. The method of claim 1, further comprising administering a therapeutically-effective amount of a second therapeutic agent.

21. The method of claim 20, wherein the second therapeutic agent is an antiviral agent.

22. The method of claim 20, wherein the second therapeutic agent is an antibiotic.
